(19) United States
(12) Patent Application Publication
(10) Pub. No.: US 2013/0030282 A1
(43) Pub. Date: Jan. 31, 2013

(54) SYNTHESIS AND CHARACTERIZATION OF NEAR IR FLUORESCENT MAGNETIC AND NON-MAGNETIC ALBUMIN NANOPARTICLES FOR BIOMEDICAL APPLICATIONS

(71) Applicants: Shlomo Margel, Rehovot (IL); Sarit Cohen, Peduel (IL); Enav Corem Salkmon, Ness Ziona (IL); Michal Pellach, Givat Shmuel (IL)

(72) Inventors: Shlomo Margel, Rehovot (IL); Sarit Cohen, Peduel (IL); Enav Corem Salkmon, Ness Ziona (IL); Michal Pellach, Givat Shmuel (IL)

(73) Assignee: BAR ILAN UNIVERSITY, Ramat Gan (IL)

(21) Appl. No.: 13/550,937

(22) Filed: Jul. 17, 2012

Related U.S. Application Data
(60) Provisional application No. 61/457,954, filed on Jul. 18, 2011.

(51) Int. Cl. A61K 49/00; A61K 49/14; A61K 9/14; A61K 47/42; A61B 6/00; A61B 5/055; A61B 6/03; A61B 8/00; A61K 49/04; A61K 49/18; B82Y 40/00; B82Y 5/00

(52) U.S. Cl. 600/411; 424/9.6; 424/9.411; 424/9.42; 424/9.4; 424/9.322; 424/491; 514/776; 600/431; 600/427; 977/773; 977/795; 977/895; 977/927

(57) ABSTRACT

The present invention discloses Near Infrared (NIR) fluorescent albumin nanoparticles having a structure selected from a core structure or a core-shell structure. Also disclosed are a process of preparing these NIR fluorescent albumin nanoparticles, and a method of in vivo detection of pathologies, in particular cancer pathology, by using administering these NIR fluorescent albumin nanoparticles to a patient.

### FIELD OF INVENTION

The present invention discloses nanometer-sized albumin particles, which are suitable for use in biomedical imaging, diagnostics and therapy. In particular, the present invention discloses near-infrared (NIR) fluorescent nanometer-sized albumin particles for use in cancer early detection and therapy, and a corresponding method for detecting pathology of cancer.

### BACKGROUND OF THE INVENTION

Fluorescent nanoparticles have attracted much interest in the fields of biological imaging, biosensing and bioanalysis due to their various advantages over single dye molecules. For example, besides the simple additive effect achieved by confining a large number of fluorescent molecules into a small volume, increasing the fluorescence per particle, dye molecules entrapped within matrix particles have a matrix shielding effect from molecular oxygen, which reduces formation of reactive oxygen species (ROS) hence reducing photobleaching. The particle matrix also reduces mobility of the dye molecules reducing the chance of collision, thus reducing fluorescence quenching, giving overall increased photostability.

In fluorescence imaging, the energy from an external source of light is absorbed by the imaging agent injected near the tumor site. The main barriers to optical imaging of a tissue are high light scattering, autofluorescence and high absorption by hemoglobin (Hb) in the mid-visible band. Thus, depending upon the wavelength of light employed (typically dependent on the fluorophore used), different penetration depths can be achieved. For instance, UV-vis spectral range photons are strongly absorbed by the most relevant tissue chromophores, deoxy- and oxyhemoglobin (HbO₂), within the first few micrometers to a millimeter of tissue thickness, thus limiting its penetration in the IR region >1150 nm where there is high absorbance of water.

In recent decades, in vivo fluorescence imaging has experienced substantial growth with the “opening” of the Near-infrared (NIR) “window”. Near-infrared light of 700 to 1000 nm achieves the highest tissue penetration due to minimal absorbency of the surface tissue in this spectral region.

Albumin is the most abundant plasma protein (35-50 g/L human serum) and with a molecular weight of approximately 66.5 kDa and an effective diameter of 7.2 nm, it is one of the smallest plasma proteins. It is readily available, biodegradable, has a long half-life in vivo and lacks toxicity and immunogenicity. When Human serum albumin (HSA) is broken down, the amino acids therein provide nutrition to peripheral tissue. Also, albumin is acidic, very soluble, and stable at the pH range of 4-9. These properties, as well as its preferential uptake in tumor and inflamed tissue, make it an ideal candidate for drug delivery. HSA and bovine serum albumin (BSA) have similar properties, due to structural similarities between them.

Albumin has been found to show an “enhanced permeability and retention” (EPR), a term used to describe a “leaky” capillary combined with an absent or defective lymphatic drainage system.

In addition to the EPR effect, it has been proposed that albumin is a major energy and nutrition source for tumor growth.

There is also evidence for active targeting of albumin. For example, preliminary evidence suggests that binding of albumin to SPARC (secreted protein acid rich in cysteine), an extracellular matrix glycoprotein that is overexpressed and associated with poor prognosis in a variety of cancers, can also initiate transcytosis.

Conjugation of therapeutic peptides or cytokines with albumin, has therefore become an attractive approach as an alternative to drug targeting. Indeed, Albumin nanoparticles for cancer treatment are already commercially available as drug carriers of Paclitaxel (Abraxane) [Hawkins, Soon-Shiong et al., Adv. Drug Del. Rev., 2008, 60(8) 876-885].

“Evans blue” is an azo dye with four sulphonate groups, with excitation peaks at 470 nm and 540 nm, and an emission peak at 680 nm. “Evans blue” binds rapidly and tightly to circulating albumin and makes subcutaneously growing tumors turn blue after injection. Anionic cyanine dyes containing two sulfonate groups, such as 3,3′-di-(γ-sulfopropyl)-4,5,4′,5′-dibenzo-9-ethylthiacarbocyanine betaine and 3,3′-di-(γ-sulfopropyl)-9-methylthiacarbo-cyanine betaine, have also been shown to efficiently interact with both HSA and BSA. Despite this, other studies have shown a decrease in binding of dyes to albumin with an increasing number of sulphonate groups, and that increased hydrophobicity increases binding.

Fluorescent NIR-absorbing nanoparticles described until now have been either NaYF4:Yb,Er upconverting nanoparticles that emit in the visible region of the spectrum, gold nanoclusters entrapped in BSA loaded into silica nanoparticles, HSA coated gold nanostructures or quantum dots. Alternatively, some exemplary fluorescent NIR-absorbing nanoparticles are covalently attached to the particle surface. However, while fluorescent dyes, including NIR dyes have been attached to the surface of albumin particles (Sowell J. et al., J. of Chromatography B, 2001, 755: 91-99), this method of attaching the dye is less desirable since it does not allow to protect the dye from the surrounding and also changes the surface chemistry and properties of the particles.

Thus, although soluble albumin has been shown to attach (at its surface) to agents such as NIR-dyes, there are to date no albumin nanoparticles containing entrapped NIR cyanine dyes that can be used to selectively detect cancer in vivo and/or to combine detection in vivo along with treatment.

In particular, there is a long and felt need to find novel slow-release biodegradable nanoparticles that can selectively bind to cancer cells and can have fluorescent properties in the NIR range, thereby enabling them to be used as a highly sensitive detection tools as well as a therapeutic measure.

### SUMMARY OF INVENTION

According to one aspect of the invention, there is provided Near Infrared (NIR) fluorescent albumin nanoparticles having a structure selected from:

a. A core structure, the core comprising at least one NIR dye encompassed within albumin nanoparticles and optionally comprising a dyed or non-dyed contrast agent;

b. A core-shell structure, the core comprising at least one material selected from a dyed or non-dyed metal or metal oxide, a dyed or non-dyed contrast agent, and a dyed or non-dyed organic compound having a hydrophilic surface, wherein the core is coated by a shell comprising one or more layers of albumin encompassing at least one NIR dye within it.

According to preferred embodiments of the invention, the NIR dye is a cyanine dye. Preferably, the cyanine dye is a dye absorbing in the range of 700-1000 nm. Also preferably, the cyanine dye is selected from ICG, IR-820, IR-806, IR-783, IR-786, DTTCI, Cy7, cypate derivatives thereof and carboxylic acid derivatives thereof (CANIR).

According to additional preferred embodiments of the invention, the contrast agent is an X-ray contrast agent, or a CT contrast agent, selected from iron oxide, gold (Au), Barium compounds and Bismuth compounds.

According to more preferred embodiments of the invention, the MRI-contrast agent is selected from iron oxide, Cobalt, Nickel and ferro-fluid.

According to yet additional preferred embodiments of the invention, the organic compound is an organic polymer. Preferably, the organic polymer is selected from polystyrene, poly(methyl methacrylate) (PMMA) and derivatives thereof.

According to some more preferred embodiments of the invention, the NIR albumin nanoparticles described herein, contain an additional non-dyed albumin external coating layer.

According to additional preferred embodiments of the invention, the NIR albumin nanoparticles described herein have a diameter ranging from 1 nm to 1000 nm.

According to yet additional preferred embodiments of the invention, the NIR albumin nanoparticles described herein further encompass at least one bioactive agent, the bioactive agent being selected from a targeting agent, a cancer drug and combinations thereof. Preferably, the targeting agent is selected from a protein, a peptide, an antibody, a small molecule, an oligonucleotide, a morpholino oligonucleotide, a peptide nucleic acid, or a drug. Also preferably, the bioactive agent is selected from peanut agglutinin (PNA), EGF, uMUC-1, antiCEA, V8, antiTAG-72, TNF-related apoptosis-inducing ligand (TRAIL), folic acid, doxorubicin, methatroxate and taxol.

Preferably, the bioactive ligand is attached to the NIR albumin nanoparticles via a spacer molecule.

According to another aspect of the invention, there is provided a process for the production of NIR albumin nanoparticles, the process comprising:

a. interacting at least one NIR dye with albumin, thereby forming a physical complex of the albumin and the dye, and

b. either precipitating the physical complex by the addition of a denaturating agent in an aqueous phase, or crosslinking the physical complex with a crosslinker.

According to one preferred embodiment of the invention, the precipitating is conducted at a temperature ranging from 30° C. to 100° C.

Preferably, the denaturating agent is an alcohol selected from ethanol, ethylene glycol, and mixtures thereof.

According to additional preferred embodiments of the invention, the process described herein, further contains in the aqueous continuous phase one or more contrast agents and/or drugs.

According to yet additional preferred embodiments of the invention, the crosslinking is conducted at a temperature ranging from 4° C. to 100° C.

According to more preferred embodiments of the invention, the crosslinker is a polyaldehyde. Preferably, the polyaldehyde is glutaraldehyde.

According to some more preferred embodiments of the invention, the process further includes adding a non-dyed albumin coating on the albumin core nanoparticles or on the NIR dyed core shell nanoparticles, by precipitating albumin thereon.

According to additional preferred embodiments of the invention, the process further includes binding at least one bioactive agent to an outer albumin layer of the albumin nanoparticles, by activation of at least one functional groups on the albumin, the functional group selected from carboxylates, amines, thiols and hydroxyls, thereby obtaining activated nanoparticles, followed by interaction of the activated nanoparticles with at least one bioactive agent, selected from a protein, a peptide, an antibody, an oligonucleotide or a drug.

According to additional preferred embodiments of the invention, the process comprises adding at least one bioactive agent to the aqueous phase, thereby forming a physical complex of the albumin and the dye, containing at least one bioactive agent trapped within, wherein the bioactive agent is selected from a protein, a peptide, an antibody, an oligonucleotide or a drug.

Preferably, the bioactive agent is selected from peanut agglutinin (PNA), EGF, uMUC-1, antiCEA, V8, antiTAG-72, TNF-related apoptosis-inducing ligand (TRAIL), folic acid, doxorubicin, methatroxate and taxol.

According to additional preferred embodiments of the invention, the bioactive agent is attached to the albumin via a spacer.

According to yet another aspect of the invention, there is provided a method of in-vivo detecting of pathology by collecting fluorescent light emitted from a tissue binded to the Near Infrared (NIR) fluorescent albumin nanoparticles of claims 1-14, the method comprising:

a) administering to a patient the Near Infrared (NIR) fluorescent albumin nanoparticles of claims 1-14,

b) administering to a patient an in-vivo sensing device, comprising at least one illumination source, an optical system and a light sensor;

c) illuminating in-vivo tissue external to the in-vivo sensing device; and

d) collecting fluorescent light reflected from the tissue onto the light sensor by using the optical system.

According to some preferred embodiments of the invention, the Near Infrared (NIR) fluorescent albumin nanoparticles contain at least one contrast agent, and the method further includes using at least one more detection method, selected from magnetic resonance imaging (MRI), CT imaging, optical imaging, ultrasound imaging, paraCEST imaging or a combination thereof.

According to additional preferred embodiments of the invention, the Near Infrared (NIR) fluorescent albumin nanoparticles contain at least one bioactive agent, the bioactive agent being a drug, and the method further comprising treating the pathology by releasing the drug of the Near Infrared (NIR) fluorescent albumin nanoparticles.

Preferably, the pathology is a pathology of cancer and cancer related diseases.

More preferably, the pathology is a pathology of cancer and cancer related diseases and the bioactive agent is a therapeutic agent used in the treatment or prevention of cancer, or in the alleviation of symptoms associated with cancer.

According to additional preferred embodiments of the invention, administering the Near Infrared (NIR) fluorescent albumin nanoparticles is conducted orally.

According to yet additional preferred embodiments of the invention, the Near Infrared (NIR) fluorescent albumin nanoparticles and/or the in-vivo sensing device are in a form of a capsule suitable for detecting pathology in the gastrointestinal (GI) tract during its passage through the GI tract.

### BRIEF DESCRIPTION OF THE DRAWINGS

FIG. 1 is a graph exhibiting the absorption (A) and fluorescence emission (B) spectra of free CANIR dye and the CANIR-HSA nanoparticles;

FIG. 2 is a graph exhibiting the photostability of the CANIR-HSA nanoparticles (A) and free CANIR (B) as a function of time;

FIG. 3 is a graph exhibiting the interaction between the non-conjugated (A) and the PNA-conjugated (B) fluorescent albumin nanoparticles and the human colonic cancerous cell lines: LS174t, HT29 and SW480 implanted on chicken CAM;

FIG. 4 is a graph exhibiting the interaction between the non-conjugated (A) and anti-CEA antibodies-conjugated (B) fluorescent albumin nanoparticles and the human colonic cancerous cell lines: LS174t and HT29 implanted on chicken CAM;

FIG. 4C is a graph exhibiting non-pathological CAM treated with both the non-conjugated and the anti-CEA antibodies-conjugated NIR fluorescent HSA nanoparticles;

FIG. 5 is a graph exhibiting the fluorescence intensities of specific labeling of tumor implants in chicken embryo with core-shell IO nanoparticles (IO-NIR-HSA);

FIG. 6 is a graph exhibiting color photographs and logarithmically scaled fluorescent images of LS174t tumor cell line from a typical mouse model treated with PNA-conjugated NIR fluorescent HSA nanoparticles, after a recovery of 1.5 hours (FIG. 6A) and 4 hours (FIG. 6B).

FIG. 7 is a graph exhibiting color photographs and logarithmically scaled fluorescent images of LS174t tumor cell line from a typical mouse model treated with PNA-conjugated NIR fluorescent HSA nanoparticles, after a recovery of 4 hours;

FIG. 8 is a graph exhibiting fluorescent and grayscale images of LS174t (A) and HT29 (B) colon tumor cell lines treated with non-conjugated (1) and anti-CEA (2) and anti-TAG-72 (3) antibodies-conjugated NIR fluorescent HSA nanoparticles;

FIG. 8C is a graph exhibiting anti-CEA and anti-TAG-72 antibodies-conjugated NIR fluorescent HSA nanoparticles in the colons of the healthy mice;

FIGS. 8A4 and 8B4 are graphs exhibiting the auto-fluorescence signal of the non-treated tumor cell lines;

FIG. 9 is a graph exhibiting for example the histological analysis of lumen-facing and non-lumen-facing tumors;

FIG. 10 is a graph exhibiting mouse colon tumors labeled with dyed core-shell NIR-HSA polystyrene nanoparticles;

FIG. 11 is a graph exhibiting mouse colon tumors labeled with NIR iron-oxide nanoparticles conjugated to PNA; and

FIG. 12 is a graph exhibiting a fluorescent image of human pathological and non-pathological colon tissue on a microscope slide.

### DETAILED DESCRIPTION OF THE INVENTION

The present invention describes nanometer-sized near-infrared (NIR) fluorescent albumin particles, and the in vivo use thereof in biomedical imaging, diagnostics and therapy.

The inventors have successfully designed and prepared albumin nanoparticles containing within them a variety of cyanine dyes, such as indocyanine green (ICG), IR783 and various derivatives of these dyes. These dyes are entrapped by strong non-covalent interactions.

It should be noted that the nanoparticle of the present invention may be structured either in a “core structure” or in a “core-shell” structure.

Generally, the term “core structure” means a structure only formed of a core without a shell, whereas a “core-shell” structure means a structure including a core and one or more shells surrounding the core.

In particular, the term “core structure”, used interchangeably with the term “albumin core nanoparticle”, refers to a core comprising of albumin encompassing NIR dyes within it. The albumin core nanoparticles may also include one or more contrast agents, e.g., iron oxide for MRI, gold nanoparticles or iodinated nanoparticles for CT imaging, etc.

The term “core-shell structure”, used interchangeably with the term “albumin core-shell nanoparticle”, refers to a core coated with one or more layers of the albumin encompassing NIR dyes within it, such that the core itself is made of a variety of additives, such as a metal, a contrast agent, an organic compound etc. In this case, the core can comprise either dyed or non-dyed materials.

Thus, according to one aspect of the invention, there are provided Near Infrared (NIR) fluorescent albumin nanoparticles having a structure selected from:

a. A core structure, the core comprising of at least one NIR dye encompassed within albumin nanoparticles and optionally comprising a dyed or non-dyed contrast agent;

b. A core-shell structure, the core comprising at least one material selected from a dyed or non-dyed metal or metal oxide, a dyed or non-dyed contrast agent, and a dyed or non-dyed organic compound having a hydrophilic surface, wherein the core is coated by a shell comprising one or more layers of albumin encompassing at least one NIR dye within it.

The term “nanoparticles” as used herein refers to particles with an average diameter ranging from a few nanometers up to 3000 nm.

According to one preferred embodiment, The NIR albumin nanoparticles described herein have a diameter ranging from 1 nm to 1000 nm.

Particle size and size distributions of the albumin nanoparticles described herein (including albumin-coated core-shell nanoparticles as described further below) may be controlled by variation of various parameters involved in their preparation.

The term “albumin” as used herein interchangeably with the terms “albumin particles” and/or “albumin nanoparticles” and includes albumin particles prepared from human source or from animal source, depending on the need.

The term “Near Infrared (NIR) fluorescent nanoparticles” as used herein refers to albumin nanoparticles containing NIR fluorescent dyes and exhibiting fluorescence.

The amount of albumin in the aqueous continuous phase ranges from about 1 weight percent to about 10 weight percent.

The amount of dye ranges from about 0.01 weight percent to about 10 weight percent, relative to albumin.

Since the in vivo biological window is between 650-1000 nm, it is preferable to use a fluorescent material that emits NIR fluorescence in this wavelength range.

As noted hereinabove, according to scientific literature, NIR dyes such as Indocyanine Green (ICG) and other structurally related cyanine dyes have been shown to have high affinity to albumin (HSA, BSA, etc.). Indeed, it has been shown by the present inventors that the preparation of albumin nanoparticles containing ICG, IR783 as well as various derivatives of these dyes, entrapped by strong non-covalent interactions, was followed by no leakage of the entrapped NIR dye into the continuous phase, e.g., PBS, PBS containing 4% albumin or human bowel juice.

Thus, according to one preferred embodiment, the NIR dye used in the NIR albumin nanoparticles described herein, is a cyanine dye.

The term “cyanine dye” as used herein refers to a fluorogenic compound that comprises 1) a substituted or unsubstituted benzazolium moiety, 2) a polymethine bridge and 3) a substituted or unsubstituted pyridinium or quinolinium moiety. These monomer or dye moieties are capable of forming a non-covalent complex with nucleaic acid and/or albumin and demonstrating an increased fluorescent signal after formation of the nucleic acid-dye complex or an albumin-dye complex.

The cyanine dye can be either an anionic cyanine dye, a neutral cyanine dye or a cationic cyanine dye.

The term “anionic cyanine dye” as used herein, refers to a cyanine dye, as defined hereinabove, that exhibits a negative charge at physiological pH.

The term “neutral cyanine dye” as used herein, refers to a cyanine dye, as defined hereinabove, that exhibits no charge at physiological pH.

The term “cationic cyanine dye” as used herein, refers to a cyanine dye, as defined hereinabove, that exhibits a positive charge at physiological pH, such as IR 786 or DTTCI.

Yet more preferably, the cyanine dye suitable for use in the present invention absorbs in the range of 700 nm to 1000 nm.

One preferable example of a suitable NIR dye is indocyanine green (ICG), a water-soluble tricarbocyanine dye approved by the FDA for use in vivo. ICG has a peak spectral absorption at 790 nm and an emission at 830 nm.

Cyanine 7 (marked as Cy7) is another fluorescent dye that belongs to the Cyanine family of synthetic polymethine dyes. Cy7 is water-soluble and has an absorbance maximum of 747 nm and an emission maximum of 776 nm, which is in the near IR.

Other commercially available structurally related dyes include IR-820 (2-[2-[2-Chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt), IR-806 (2-[2-[2-chloro-3-[2-[1,3-dihydro-3,3-dimethyl-1-(4-sulfobutyl)-2H-indol-2-ylidene]-ethylidene]-1-cyclopenten-1-yl]-ethenyl]-3,3-dimethyl-1-(4-sulfobutyl)-3H-indolium hydroxide, inner salt, sodium salt), IR-783 (2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide, inner salt sodium salt), IR-786 (2-(2-[2-Chloro-3-([1,3-dihydro-1,3,3-trimethyl-2H-indol-2-ylidene]ethylidene)-1-cyclohexen-1-yl]ethenyl)-1,3,3-trimethylindolium perchlorate) and N,N-diethyl-2,2-dithiaditricarbocyanine iodide (DTTCI).

Various other suitable derivatives have been synthesized, and among them are cypate and carboxylic acid derivatives of IR-806 and of IR-783 (CANIR).

Scheme 1 below depicts the structures of selected NIR fluorescent cyanine dyes

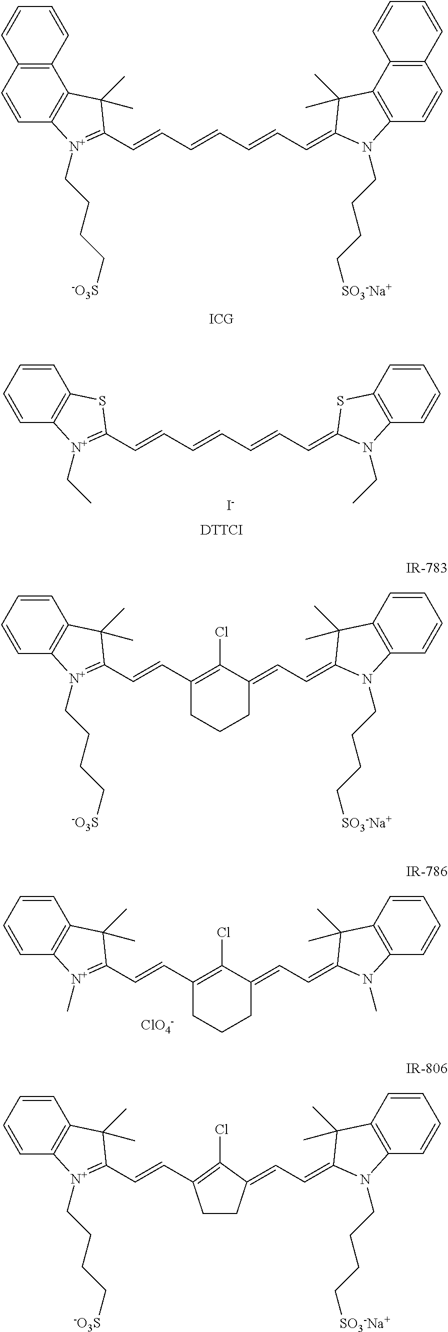

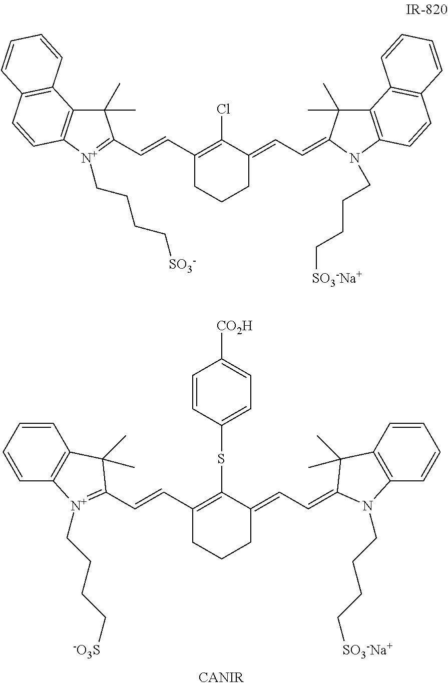

Thus, according to another preferred embodiment, the NIR albumin nanoparticles described herein are selected from the group comprising of ICG, IR-820, IR-806, IR-783, IR-786, DTTCI, Cy7, cypate derivatives thereof and carboxylic acid derivatives thereof (CANIR).

It is important to note that the present invention encompasses NIR fluorescent dyes which are entrapped within the nanoparticles, as described in further details below.

The particles may also be of core-shell structure of any type, with either a biodegradable core (e.g. iron oxide containing nanoparticles) or non-biodegradable core (e.g. crosslinked or non-crosslinked polystyrene), with the NIR-dyed albumin coating, wherein the albumin is entrapped within the albumin matrix, thus forming the shell.

Often, an additional external albumin coating in the absence of the dye was performed. This was in order to ensure that the dye was within the coating, and not exposed to a low extent on the particle surface, which could result in nanoparticle aggregation, resulting from the high affinity of dye towards HSA. This additional albumin coating also preserves the Z-potential of the albumin and increases the surface functional groups (carboxylate, amine, hydroxyl and thiol) concentration for physical or covalent conjugation of bioactive agents (e.g., drugs, targeting reagents, peptides, proteins, etc.) reagents.

As noted above, a core-shell structure is often developed to include additional active components into the composition.

For example, in order to enhance the detection properties of the NIR nanoparticles of the invention, the core may contain one or more metal or metal oxides and/or one or more contrast agents. This enables the use of more than one detection method (i.e. NIR fluorescence and MRI or CT), thereby increasing both the accuracy and the reliability of the detection.

As noted hereinbelow various metals and/or metal oxides can be used within the core. Preferably, the metal oxide is iron oxide, although other oxides may be used.

As used herein, the term “contrast agent” refers to any agent which is detectable by any means. Examples of contrast agents include, but are not limited to, MRI contrast agents, CT contrast agents, X-ray contrast agents, nucleosan contrast agents, and ultrasonic contrast agents, among others.

Thus, according to one preferred embodiment of the invention, the structure of the NIR albumin nanoparticles is a core-shell structure, having at least one contrast agent, this agent being an X-ray contrast agent, or a CT contrast agent.

More preferably, this X-ray agent and/or this CT contrast agent is selected from iron oxide, gold (Au), Barium compounds and Bismuth compounds.

As noted above, the NIR fluorescent albumin nanoparticles may also entrap ferro-fluid, thereby becoming also magnetic, and being suitable for MRI detection. Thus, according to yet another preferred embodiment, the contrast agent is an MRI contrast agent.

More preferably, this MRI-contrast agent is selected from iron oxide, Cobalt, Nickel and ferro-fluid.

Preferably, the concentration/amount of the contrast agent used herein ranges from about 0.001 weight percent to about 50 weight percent, more preferably from about 0.005 weight percent to about 20 weight percent. In particular, the range is 0.005-5% for MRI applications and 1-20% for CT applications.

As noted hereinabove, the NIR albumin nanoparticles described herein may contain in the core an organic compound that can enhance the dispersibility of the nanoparticles in vivo as well as to enhance their encapsulation capacity.

According to one preferred embodiment, this organic compound is an organic polymer.

While any polymer which is at least slightly hydrophilic at the surface may be used for the purpose of the present invention, it has been found that preferably, the organic polymer is selected from polystyrene, poly(methyl methacrylate) (PMMA) and derivatives thereof.

Preferably, the concentration/amount of the polymer used herein ranges from about 70 weight percent to about 99.5 weight percent.

A surfactant or stabilizer is often an important additive to accompany the polymer, although stabilization of nanoparticles can also be achieved by charge in the absence of a stabilizer.

Thus, according to one preferred embodiment, this organic compound is an organic polymer coated by a surfactant or a stabilizer.

As used herein, a “surfactant” refers to any molecule containing a polar portion that thermodynamically prefers to be solvated by a polar solvent, and a hydrocarbon portion that thermodynamically prefers to be solvated by a non-polar solvent. The term “surfactant” is also meant to encompass anionic, cationic, or non-ionic surfactants. As used herein, the term “anionic surfactant” refers to a surfactant with a polar portion that ionizes to form an anion in aqueous solution. Similarly, a “cationic surfactant” refers to a surfactant having a cationic polar portion that ionizes to form a cation in aqueous solution. Likewise, a “non-ionic” surfactant refers to a surfactant having a polar portion that does not ionize in aqueous solution.

The term “stabilizer” as used herein refers to a pharmaceutical acceptable excipient, which protects the active pharmaceutical ingredient and/or the formulation from chemical and/or physical degradation during manufacturing, storage and application. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined.

While not wanting to be bound to theory, it is generally believed that a surfactant refers to a molecule that is effective to reduce a surface or an interfacial tension between a first substance dispersed in a second substance such that the first substance is solvated and any molecular groups of the first substance are dispersed. The surfactant component may be characterized on the HLB (Hydrophile-Lipophile Balance) scale that ranges from less than about 1 to more than about 13 units. A surfactant component having an HLB value of less than about 6.0 units may be described as being poorly, or not dispersable in an aqueous or water-based composition. In addition, a surfactant component having an HLB value of less than about 6.0 units may be characterized as a hydrophobic or non-ionic surfactant. A surfactant component having an HLB value of more than about 7.0 units may be described as being capable of forming a milky to translucent to clear dispersion when the surfactant having an HLB value of more than about 7.0 units is dispersed in an aqueous or water-based composition.

Preferably, the surfactant component suitable for use in the present invention facilitates the preparation of nanoparticles having a diameter ranging from a few nm up to 1 micron.

The surfactant component suitable for use in the present invention may be supplied as individual surfactants or supplied in various prepared mixtures of two or more surfactants that are subsequently combined.

Some non-exhaustive examples of suitable surfactants include, but are not limited to, gelatin, polyvinypyrrolidone, SDS, Pluoronic 123, PEG-PPO copolymers, albumin, proteins, etc., or any combination thereof.

Preferably, the concentration/amount of the surfactant and/or stabilizer used herein ranges about 0.001 weight percent to about 20 weight percent, more preferably from about 0.01 weight percent to about 10 weight percent.

As can be seen in the Examples section which follows, polystyrene core nanoparticles with diameters of 500-1000 nm and of narrow-size distribution were synthesized by dispersion polymerization. Nanoparticles below 500 nm were synthesized by emulsion polymerization. A modified miniemulsion polymerization method may also be applied, in the presence of a fluorescent material. Encapsulation of dye into template polystyrene particles was performed using a swelling and evaporation method, giving fluorescent polystyrene particles. The nanoparticles were then functionalized by coating the PS with albumin. Crosslinking the albumin (e.g. with glutaraldehyde) was used at times to make it less prone to digestion by enzymes of the gastrointestinal tract (GIT), which requires that the particles be non-toxic and bio compatible.

While albumin is used as both an active and passive targeting agent, specificity of the particles, for example selectivity towards tumor cells, may be increased by additional conjugation of targeting agents. These include numerous antibodies, proteins and small peptides with corresponding receptors that are upregulated on tumor cells as compared to non-cancerous cells. Examples include, but are not limited to, peanut agglutinin (PNA); epidermal growth factor (EGF); underglycosylated mucin-1 antigen (uMUC-1); carcinoembryonic antigen monoclonal antibody (antiCEA); Tumor associated glycoprotein-72 monoclonal antibody (antiTAG-72); TNF-related apoptosis-inducing ligand (TRAIL), and single chain FV fragment (V8).

As can be seen in the experimental section which follows, these NIR-dyed albumin containing nanoparticles were used as a model for targeting colonic neoplasms both actively, where a specific targeting agent was covalently conjugated to the particles, and passively, where the albumin itself was exploited as a “nutrient” for neoplasms that generally have increased metabolism and thus increased propensity to ingest proteinaceous substances, compared to non-cancerous cells. Each type of particles—with or without conjugation to a targeting agent, and with or without a spacer arm, was found to specifically label polyps and cancerous tumors. However, the specificity of the bioactive-conjugated fluorescent albumin nanoparticles (wherein the bioactive agents were homing reagents) towards the tumor was often higher than that of the non-conjugated albumin nanoparticles.

This phenomenon was demonstrated in tumor implants in chicken embryo, mouse and rat models. The main advantage of these particles, beside the non-immunogenicity, non-toxicity and biodegradability, is their fluorescence in the NIR region of the electromagnetic spectrum, allowing in vivo imaging with low tissue absorbance, increased tissue penetration and low autofluorescence of bodily tissues. This allows for early detection and therapy of neoplasms in the gastrointestinal tract. The work with the colonic neoplasms was used as a model for cancer detection and therapy. However, other kinds of cancer can also be detected and treated with these fluorescent non-conjugated or bioactive-conjugated nanoparticles.

Thus, according to yet another embodiment of the invention, the NIR albumin nanoparticles described herein further encompass at least one bioactive agent.

The term “bioactive agent”, used interchangeably with the terms “bioactive reagent” and “bioactive ligand”, as used herein, is used in its broadest sense and includes any substance or mixture of substances that have clinical use, as described hereinabove. Consequently, bioactive agents may or may not have pharmacological activity per se, e.g., a dye or a detection molecule. A single bioactive agent may be utilized or, in alternate embodiments, a variety of bioactive agents may be incorporated into the nanoparticles of the present invention.

As indeed shown hereinbelow, the NIR albumin nanoparticles described herein further encompass at least one bioactive agent, this bioactive agent being preferably selected from a targeting agent, a cancer drug and combinations thereof.

According to one specific embodiment, the bioactive agent is a targeting agent.

As used herein, the term “targeting agent”, is used interchangeably with the terms “homing agent”, means any agent having the ability to direct a moiety associated with the targeting agent to the surface of a cell, to the surface of a particular type of cell, or to the nucleus of a cell.

A targeting agent can comprise, but is not limited to, proteins, peptides, antibodies, small molecules, oligonucleotides, morpholino oligonucleotides, peptide nucleic acids and drugs.

Antibodies, particularly monoclonal antibodies, may also be used as site-targeting ligands. Immunoglobin-γ (IgG) class monoclonal antibodies have been conjugated to liposomes, emulsions and other microbubble particles to provide active, site-specific targeting. Generally, these proteins are symmetric glycoproteins (MW ca. 150,000 Daltons) composed of identical pairs of heavy and light chains. Hypervariable regions at the end of each of two arms provide identical antigen-binding domains. A variably sized branched carbohydrate domain is attached to complement-activating regions, and the hinge area contains particularly accessible interchain disulfide bonds that may be reduced to produce smaller fragments.

Preferably, monoclonal antibodies are used in the antibody compositions of the invention. Monoclonal antibodies specific for selected antigens on the surface of cells may be readily generated using conventional techniques. Hybridoma cells can be screened immunochemically for production of antibodies specifically reactive with an antigen, and monoclonal antibodies can be isolated. Other techniques may also be utilized to construct monoclonal antibodies.

Within the context of the present invention, antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)2) and recombinantly produced binding partners, single domain antibodies, hybrid antibodies, chimeric antibodies, single-chain antibodies, human antibodies, humanized antibodies, and the like.

The targeting agents may include polypeptides, which similar to antibodies, may have high specificity and epitope affinity for use as vector molecules for targeted contrast agents. These may be small oligopeptides, having, for example, 5 to 20 amino acids, specific for a unique receptor sequences or larger, biologically active hormones such as cholecystokinin. Smaller peptides potentially have less inherent immunogenicity than nonhumanized murine antibodies. Peptides or peptide (nonpeptide) analogues of cell adhesion molecules, cytokines, selectins, cadhedrins, Ig superfamily, integrins and the like may be utilized for targeted imaging and/or therapeutic delivery.

In some instances, the ligand is a non-peptide organic molecule. The term “non-peptide ligands” refers to moieties which are commonly referred to as “small molecules” lacking in polymeric character and characterized by the requirement for a core structure other than a polymer of amino acids. The non-peptide ligands useful in the invention may be coupled to peptides or may include peptides coupled to portions of the ligand which are responsible for affinity to the target site, but it is the non-peptide regions of this ligand which account for its binding ability.

Carbohydrate-bearing lipids may be used as targeting agents.

The targeting agent can be of any size, as long as it retains its ability to direct a moiety associated with the targeting agent to the surface of a cell or to the surface of a particular type of cell.

In particular, since the NIR albumin nanoparticles described herein are most suitable to detect cancer cells, it is advisable to enhance the selectivity of the NIR albumin bioparticles, by attaching to it bioactive agents that can specifically bind to tumor sites, either in a non-selective or highly-selective manner.

Some examples of cancer targeting agents include, but are not limited to, peanut agglutinin (PNA), EGF, uMUC-1, antiCEA, V8, antiTAG-72, TNF-related apoptosis-inducing ligand (TRAIL), folic acid, doxorubicin, methatroxate and taxol.

Thus, according to one preferred embodiment, the NIR albumin nanoparticles described herein have a bioactive agent which is selected from peanut agglutinin (PNA), EGF, uMUC-1, antiCEA, V8, antiTAG-72, TNF-related apoptosis-inducing ligand (TRAIL), folic acid, doxorubicin, methatroxate and taxol.

The targeting agent may be linked to the NIR albumin nanoparticles either directly or via a spacer molecule.

As can be seen in the Examples section below, each type of particles—with or without conjugation to a targeting agent, and with or without spacer arms, was found to specifically label polyps and cancerous tumors.

It has been found that it is advantageous that the bioactive targeting agent has an enhanced flexibility, for example by linking it to the nanoparticles via a spacer molecule. Thus, in one preferred embodiment, the bioactive agent is attached to the NIR albumin nanoparticles via a spacer molecule.

The term “spacer” as used herein, is used interchangeably with the terms “tether” or “arm” and refers to a typically long molecule, such as oligomer or polymer, that is covalently attached to, and interposed between the NIR nanoparticle and the bioactive agent, as an alternative to direct attachment between them.

Therefore, the spacer molecule is typically a molecule such as an bifunctional molecule, oligomer or polymer, having a molecular weight (MW) of at least 50 grams/mol, but more preferably its molecular weight is higher, namely at least 500 grams/mol. For example, it can be seen that the spacers used in the Examples section below included NHSPEG, NHSPEGNHS, NHSPEGMelimide, etc, PEG-polyethylene glycol. Other exemplary spacers may include divinylsulfone, glutaraldehyde, etc.

Since the compositions of the invention are intended to target tissues expressing a target moiety, such targeting is intended to be detected using low resolution and higher resolution imaging techniques. In one embodiment, the low resolution contrast agent comprises a radionuclide or optical imaging agent, which can be coupled to a target-specific ligand. Optionally, the low resolution contrast agent comprises a particle, such as a nanoparticle. Other types of particles include liposomes, micelles, bubbles containing gas and/or gas precursors, lipoproteins, halocarbon and/or hydrocarbon nanoparticles, halocarbon and/or hydrocarbon emulsion droplets, hollow and/or porous particles and/or solid nanoparticles. In one embodiment, the low resolution contrast agent comprises a halocarbon-based nanoparticle such as a perfluorooctyl bromide (PFOB) nanoparticle, detectable, for example, with fluorine MRI. A higher resolution contrast agent comprises a target-specific ligand, a contrast agent for magnetic resonance imaging (MRI), a CT imaging agent, an optical imaging agent, an ultrasound imaging agent, a paraCEST imaging agent, or a combination thereof, and, optionally, comprises a particle such as a nanoparticle. The low resolution and higher resolution contrast agent can be incorporated into the same particle.

A targeted low resolution contrast agent accumulates in tissues expressing the target moiety. A low resolution imaging technique identifies potential target tissues that contain an accumulation of the low resolution contrast agent. A targeted higher resolution contrast agent is administered having an analogous target as the low resolution contrast agent, which will also accumulate in the potential target tissue. If any potential target tissue is identified using the low resolution imaging technique, a higher resolution imaging technique is used to examine any identified potential target tissues at a higher resolution.

Thus, in one aspect, the invention is directed to a method for high resolution imaging, comprising: (a) administering a targeted low resolution contrast agent and a targeted higher resolution contrast agent having an analogous target as the low resolution contrast agent, and allowing each contrast agent to accumulate in a target tissue; (b) identifying the target tissue using a low resolution imaging technique to localize an accumulation of the low resolution contrast agent. If the low resolution imaging technique identifies a target tissue having an accumulation of the low resolution contrast agent, step (c) is applied, directed to obtaining a high resolution image of the target tissue using a higher resolution imaging technique to localize an accumulation of the higher resolution contrast agent, thereby allowing the generation of a higher resolution image than that obtained by the use of the low resolution contrast agent alone.

In another aspect, the invention is also directed to a method of delivering targeted contrast agents to a target tissue, comprising: (a) administering a low resolution targeted contrast agent selected from a targeted nuclear contrast agent and a halocarbon-based nanoparticle to a subject comprising said target tissue; (b) administering a higher resolution targeted contrast agent to the subject, selected from the group consisting of an MRI contrast agent, a CT contrast agent, an ultrasound contrast agent, an optical contrast agent, a paraCEST contrast agent and a combination thereof, wherein the higher resolution contrast agent has an analogous target as the low resolution contrast agent; and (c) allowing the contrast agents to accumulate in the target tissue, to thereby deliver targeted contrast agents to the target tissue. An image of the low resolution contrast agent that is bound to the targeted tissue can be obtained. In another embodiment, an image of the higher resolution contrast agent that is bound to the targeted tissue is obtained, optionally after the image of the low resolution contrast agent bound to the targeted tissue is obtained.

Thus, according to additional aspect of the invention, there is provided a method of in-vivo detecting of pathology by collecting fluorescent light emitted from a tissue targeted by the Near Infrared (NIR) fluorescent albumin nanoparticles described hereinabove, this method comprising:

a) administering to a patient the Near Infrared (NIR) fluorescent albumin nanoparticles described hereinabove,

b) administering to a patient an in-vivo sensing device, comprising at least one illumination source, an optical system and a light sensor;

c) illuminating in-vivo tissue external to the in-vivo sensing device; and

d) collecting fluorescent light reflected from the tissue onto said light sensor by using said optical system.

In-vivo detection by administering an in-vivo sensing device is described in many publications, known to a person skilled in the art. For example, WO 2010079484 (to “Given Imaging”) discloses a method of detecting in-vivo pathology by collecting fluorescent light emitted from a tissue. The method comprises administering to a patient an in-vivo sensing device comprising at least one illumination source for illuminating white light and at least one illumination source for illuminating light which causes excitation to in-vivo tissue, an optical system and a light sensor; illuminating in-vivo tissue external to the in-vivo sensing device; and collecting fluorescent light reflected from the tissue onto the light sensor by using the optical system. The method further comprises administering to a patient a binding agent with high affinity to a marker in-vivo, this binding agent comprising a fluorescent emitting molecule, wherein administering a binding agent is performed prior to the step of administering an in-vivo sensing device.

The in-vivo device may obtain fluorescent images along with images of the lumen it passes through. Depending on the device's optical design, the device may either illuminate the tissue at an alternating mode, i.e. the device may illuminate at white light alternatingly with illuminating at a wavelength that causes the tissue to excite, or the device may illuminate the tissue with the different wavelengths simultaneously. The in-vivo device may be a swallowable capsule, for example a capsule which may detect pathology in the gastrointestinal (GI) tract during its passage through the GI tract.

According to preferred embodiments of the invention, the Near Infrared (NIR) fluorescent albumin nanoparticles contain at least one contrast agent, and the method further including using at least one more detection method, selected from magnetic resonance imaging (MRI), CT imaging, optical imaging, ultrasound imaging, paraCEST imaging or a combination thereof.

Thus, the same particles can be used for multi-detection of the pathology by more than one detection method, thereby increasing its accuracy and reliability of detection.

As used herein the term “pathology” refers to symptoms, for example, structural and functional changes in a cell, tissue, or organs, which contribute to a disease or disorder.

According to preferred embodiments of the invention, the term “pathology” refers to pathology of cancer and cancer related diseases.

As used herein, the term “pathology of cancer” includes all phenomena that comprise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes.

The administration of the Near Infrared (NIR) fluorescent albumin nanoparticles and/or the in-vivo sensing device can be conducted by a number of in-vivo delivery methods.

As used herein, the term “in vivo delivery” refers to delivery of the Near Infrared (NIR) fluorescent albumin nanoparticles and/or the in-vivo sensing device by such routes of administration as oral, intravenous, subcutaneous, intraperitoneal, intrathecal, intramuscular, intracranial, inhalational, topical, transdermal, suppository (rectal), pessary (vaginal), and the like.

Preferably, the in-vivo application is conducted either rectally or orally.

Thus, according to preferred embodiments of the invention, the administering of the Near Infrared (NIR) fluorescent albumin nanoparticles is conducted orally.

More details on the various application methods are known to any person skilled in the art.

According to one preferred embodiment, the Near Infrared (NIR) fluorescent albumin nanoparticles and/or the in-vivo sensing device are in a form of a capsule suitable for detecting pathology in the gastrointestinal (GI) tract during its passage through the GI tract.

Besides the dye, the targeting agent and/or the contrast agent, all used to enhance the detection properties of the particles, the particles may also contain therapeutic agents. These particles may be chosen to be biodegradable thereby obtaining drug-releasing capability upon degradation.

Therefore, according to additional preferred embodiments of the invention, the Near Infrared (NIR) fluorescent albumin nanoparticles contain at least one bioactive agent, this bioactive agent being a drug suitable to treat a pathology, and the method further comprises treating this pathology by releasing the drug of the Near Infrared (NIR) fluorescent albumin nanoparticles.

The term “drug” or “bioactive agent” or “bioactive ligand” is defined hereinabove also include any agent that provides a therapeutic or prophylactic effect; a compound that affects or participates in tissue growth, cell growth and/or cell differentiation; a compound that may be able to invoke or prevent a biological action such as an immune response; or a compound that could play any other role in one or more biological processes. Moreover, any agent which may enhance tissue repair, limit the risk of sepsis, modulate the mechanical properties of the medical device, and/or deliver pharmaceutical agents may also be considered as a bioactive agent.

Examples of bioactive agents and alternative forms of these bioactive agents such as salt forms, free acid forms, free base forms, and hydrates include: antimicrobials (e.g., cephalosporins such as cefazolin sodium, cephradine, cefaclor, cephapirin sodium, ceftizoxime sodium, cefoperazone sodium, cefotetan disodium, cefuroxime azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium); penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium); erythromycins (e.g., erythromycin ethylsuccinate, erythromycin, erythromycin estolate, erythromycin lactobionate, erythromycin stearate, and erythromycin ethylsuccinate); and tetracyclines (e.g., tetracycline hydrochloride, doxycycline hyclate, and minocycline hydrochloride, azithromycin, and clarithromycin); analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); anesthetics; antiepileptics; antihistamines; non-steroidal anti-inflammatories (e.g., indomethacin, ketoprofen, flurbiprofen, naproxen, ibuprofen, ramifenazone, and piroxicam); steroidal anti-inflammatories (e.g., cortisone, dexamethasone, fluazacort, celecoxib, rofecoxib, hydrocortisone, prednisolone, and prednisone); cardiovascular drugs (e.g., coronary vasodilators and nitroglycerin); diagnostic agents; cholinomimetics; antimuscarinics; muscle relaxants; adrenergic neuron blockers; neurotransmitters; antineoplastics (e.g., cyclophosphamide, actinomycin, bleomycin, daunorubicin, doxorubicin hydrochloride, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); immunogenic agents; immunosuppressants (e.g., cyclosporine, azathioprine, mizoribine, and FK506 (tacrolimus)); gastrointestinal drugs; diuretics; lipids; lipopolysaccharides; polysaccharides; enzymes; non-steroidal antifertility agents; parasympathomimetic agents; psychotherapeutic agents; psychoactive drugs; tranquilizers; decongestants; sedative hypnotics (e.g., barbiturates such as pentobarbital and secobarbital); and benzodiazapines such as flurazepam hydrochloride, triazolam, and midazolam); steroids; sulfonamides; vitamins; antimalarials; anti-migraine agents (e.g., ergotamine, propanolol, isometheptene mucate, and dichloralphenazone); anti-parkinson agents (e.g., L-Dopa and ethosuximide); antitussives; bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate); anticholinergic agents (e.g., oxybutynin and ipratropium bromide); xanthines (e.g., aminophylline, dyphylline, metaproterenol sulfate, and aminophylline); mast cell stabilizers (e.g., cromolyn sodium); inhalant corticosteroids (e.g., beclomethasone dipropionate, beclomethasone dipropionate monohydrate, salbutamol, ipratropium bromide, budesonide, ketotifen, salmeterol, xinafoate, terbutaline sulfate, triamcinolone, theophylline, nedocromil sodium, metaproterenol sulfate, flunisolide, and fluticasone proprionate); angiogenic agents; anti-angiogenic agents; alkaloids; analgesics; narcotics (e.g., codeine, dihydrocodeinone, meperidine, morphine, and the like); opoid receptor antagonists (e.g., naltrexone and naloxone); anti-cancer agents; chemotherapeutic drugs; anti-convulsants; anti-emetics (e.g., meclizine hydrochloride, nabilone, prochlorperazine, dimenhydrinate, promethazine hydrochloride, thiethylperazine, and scopolamine); antihistimines (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine); anti-inflammatory agents (e.g., hormonal agents, hydrocortisone, non-hormonal agents, allopurinol, indomethacin, phenylbutzone and the like); prostaglandins and cytotoxic drugs; drugs affecting reproductive organs; estrogens; antibacterials (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antibodies; antibiotics (e.g., neomycin, streptomycin, chloramphenicol, cephalosporin, ampicillin, penicillin, tetracycline, and ciprofloxacin); anti-fungals (e.g., griseofulvin, ketoconazole, itraconizole, amphotericin B, nystatin, and candicidin); anti-virals (e.g., interferon alpha, beta or gamma, zidovudine, amantadine hydrochloride, ribavirin, and acyclovir); anticoagulants (e.g., heparin, heparin sodium, and warfarin sodium); antidepressants (e.g., nefopam, oxypertine, doxepin, amoxapine, trazodone, amitriptyline, maprotiline, phenylzine, desipramine, nortriptyline, tranylcypromine, fluoxetine, doxepin, imipramine, imipramine pamoate, isocarboxazid, trimipramine, and protriptyline); immunological agents; antiasthamatics (e.g., ketotifen and traxanox); antidiabetics (e.g., biguanides and sulfonylurea derivatives); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); antianginal agents such as beta-adrenergic blockers, calcium channel blockers (e.g., nifedipine and diltiazem), and nitrates (e.g., nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encamide, digoxin, digitoxin, mexiletine, disopyramide phosphate, procainamide, quinidine sulfate, quinidine gluconate, quinidine polygalacturonate, flecamide acetate, tocamide, and lidocaine); antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium); antigout agents (e.g., colchicine and allopurinol); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione); agents useful for calcium regulation (e.g., calcitonin and parathyroid hormone); anti-infectives (e.g., GM-CSF); steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens); progestins (e.g., methoxyprogesterone acetate and norethindrone acetate); corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate); thyroid hormones (e.g., levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); agents useful for erythropoiesis stimulation (e.g., erythropoietin); and antiulcer/antireflux agents (e.g., famotidine, cimetidine, and ranitidine hydrochloride).

Other examples of suitable bioactive agents, which may be included in the medical device include, for example, viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, α-IFN and γ-IFN); erythropoietin; nucleases; tumor necrosis factor; colony stimulating factors (e.g., GCSF, GM-CSF, MCSF); insulin; anti-tumor agents and tumor suppressors; blood proteins such as fibrin, thrombin, fibrinogen, synthetic thrombin, synthetic fibrin, synthetic fibrinogen; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); bone morphogenic proteins; TGF-B; protein inhibitors; protein antagonists; protein agonists; nucleic acids such as antisense molecules, DNA, RNA, and RNAi; oligonucleotides; polynucleotides; and ribozymes.

Other bioactive agents useful in the compositions and methods described herein include mitotane, halonitrosoureas, anthrocyclines, ellipticine, ceftazidime, oxaprozin, valacyclovir, famciclovir, flutamide, enalapril, metformin, itraconazole, gabapentin, fosinopril, tramadol, acarbose, lorazepan, follitropin, omeprazole, lisinopril, tramsdol, levofloxacin, zafirlukast, granulocyte stimulating factor, nizatidine, bupropion, perindopril, erbumine, adenosine, alendronate, alprostadil, betaxolol, bleomycin sulfate, dexfenfluramine, fentanyl, gemcitabine, glatiramer acetate, granisetron, lamivudine, mangafodipir trisodium, mesalamine, metoprolol fumarate, miglitol, moexipril, monteleukast, octreotide acetate, olopatadine, paricalcitol, somatropin, sumatriptan succinate, tacrine, nabumetone, trovafloxacin, dolasetron, finasteride, isradipine, tolcapone, enoxaparin, fluconazole, lansoprazole, pamidronate, didanosine, diclofenac, cisapride, venlafaxine, troglitazone, fluvastatin, losartan, imiglucerase, donepezil, olanzapine, valsartan, fexofenadine, adapalene, doxazosin mesylate, mometasone furoate, ursodiol, felodipine, nefazodone hydrochloride, valrubicin, albendazole, medroxyprogesterone acetate, nicardipine hydrochloride, zolpidem tartrate, rubitecan, amlodipine besylate/benazepril hydrochloride, paroxetine hydrochloride, podofilox, pramipexole dihydrochloride, quetiapine fumarate, candesartan, cilexetil, ritonavir, busulfan, flumazenil, risperidone, carbemazepine, carbidopa, levodopa, ganciclovir, saquinavir, amprenavir, sertraline hydrochloride, clobustasol, diflucortolone, halobetasolproprionate, sildenafil citrate, chlorthalidone, imiquimod, simvastatin, citalopram, irinotecan hydrochloride, sparfloxacin, efavirenz, tamsulosin hydrochloride, mofafinil, letrozole, terbinafine hydrochloride, rosiglitazone maleate, lomefloxacin hydrochloride, tirofiban hydrochloride, telmisartan, diazapam, loratadine, toremifene citrate, thalidomide, dinoprostone, mefloquine hydrochloride, trandolapril, mitoxantrone hydrochloride, tretinoin, etodolac, nelfinavir mesylate, indinavir, nifedipine, cefuroxime, and nimodipine.

Specific agents within these classes are within the purview of those skilled in the art and are dependent upon such factors as, for example, the type of nanoparticles in which it is utilized.

According to preferred embodiments of the invention, the bioactive agent is a therapeutic agent used in the treatment or prevention of cancer, or in the alleviation of symptoms associated with cancer.

For example, these agents may preferably include antineoplastic drugs, analgesics, genes, siRNA, contrast agents for CT and/or MRI, etc. and any combinations thereof.

As can be understood, the NIR-dye albumin nanoparticles prepared according to the present invention have been shown to successfully detect cancer in its early stages, and the same system was also used to transfer therapeutic agents to specific sites, i.e. tumors.

Thus, according to another aspect of the invention, there is provided a use of the NIR albumin nanoparticles prepared as described hereinabove, for biomedical applications.

The term “biomedical applications” as used herein refers to detection, prevention and therapy.

In particular, the present invention is most suitable for the detection, prevention and therapy of cancer and cancer related disease.

The term “cancer” refers to a proliferative disorder associated with uncontrolled cell proliferation, unrestrained cell growth, and decreased cell death/apoptosis. Cancer includes, but is not limited to, breast cancer, prostate cancer, lung cancer, kidney cancer, thyroid cancer, melanoma, follicular lymphomas, carcinomas with p53 mutations, and hormone-dependent tumors, including, but not limited to, colon cancer, cardiac tumors, pancreatic cancer, retinoblastoma, glioblastoma, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, Kaposi's sarcoma, ovarian cancer, leukemia (including acute leukemias (for example, acute lymphocytic leukemia, acute myelocytic leukemia, including myeloblastic, promyelocytic, myelomonocytic, monocytic, and erythroleukemia)) and chronic leukemias (for example, chronic myelocytic (granulocytic) leukemia and chronic lymphocytic leukemia), myelodysplastic syndrome polycythemia vera, lymphomas (for example, Hodgkin's disease and non-Hodgkin's disease), multiple myeloma, Waldenstrom's macroglobulinemia, heavy chain diseases, and solid tumors including, but not limited to, sarcomas and carcinomas such as fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, and menangioma. The terms “metastasis” and “cancer metastasis” are used interchangeably herein to refer to the ability of a cancer cell to spread to other tissues. For example, “metastasis to bone” refers to the ability of certain types of cancer including, but not limited to, breast, prostate, lung, kidney, thyroid, and melanoma, to metastasize to bone.

The term “tumor” as used herein refers to any tumor such as, without being limited to, brain tumors, preferably glioma, colon cancer, lung cancer, breast cancer, prostate cancer, bladder cancer, kidney cancer, ovarian cancer, melanoma, leukemia or multiple myeloma, preferably glioma, and metastases thereof.

Preferably, the pathology is a pathology in the gastrointestinal (GI) tract.

As shown by the present inventors, the nanoparticles described herein to be used in diagnostics, may be further used for therapy by the inclusion of suitable cancer drugs, such as paclitaxel, methotrexate, doxorubicin or 5-fluorouracil among others, or alternatively, analgesics, genes, siRNA, etc. or various combinations that can be encapsulated within the nanoparticles for site-directed therapy. The particles have the advantages of being both biodegradable and having drug-releasing capability upon degradation. The resulting nanoparticles therefore have a dual function: tumor recognition as well as site-directed therapy.

Thus, according to preferred embodiments of the invention, there is provided a use of the NIR albumin nanoparticles prepared as described hereinabove, for biomedical applications, wherein these biomedical applications are early cancer detection and/or cancer diagnostics and/or cancer therapy.

Polymeric nano-scaled particles of narrow size distribution are commonly formed by controlled precipitation methods or heterogeneous polymerization techniques, e.g., by optimal emulsion or inverse emulsion polymerization methods.

Similarity, as detailed in the experimental section which follows, the nanoparticles of the present invention were prepared by first interacting at least one NIR dye with albumin, thereby forming a physical complex of the albumin and the dye. Then, this complex was either precipitated by the addition of a denaturating agent, or it was crosslinked with a polyaldehyde.

Thus, according to another aspect of the invention, there is provided a process for the production of NIR albumin nanoparticles, this process comprising:
a. interacting at least one NIR dye with albumin, thereby forming a physical complex of the albumin and the dye, and
b. either precipitating the physical complex by the addition of a denaturating agent in an aqueous phase, or crosslinking the physical complex with a polyaldehyde.

The term “physical complex” as used herein refers to non-covalent bonding of the albumin and the dye.

The precipitation is preferably conducted by using a denaturating agent.

The term “denaturing agent” or “denaturant,” as used herein, refers to any compound or material which will cause a reversible unfolding of a protein, such as albumin.

Preferably the denaturing agent is an alcohol selected from ethanol, ethylene glycol, and mixtures thereof.

Generally, the precipitation was conducted at a temperature ranging from 30° C. to 100° C.

For example, the precipitation can be conducted by adding ethanol to an aqueous solution of albumin and a NIR dye. The obtained solution is then stirred at room temperature for some time, raising the temperature to about 70° C. and stirring for additional few hours, followed by another slight increase to about 80° C. for 12 hours. The obtained NIR fluorescent particles dispersed in water were then washed from impurities by gel filtration. Non-fluorescent HSA nanoparticles are prepared similarly to those of the NIR fluorescent HSA nanoparticles, in the absence of the CANIR dye.

The crosslinking is conducted at a temperature ranging from 4° C. to 100° C.

Examples of suitable crosslinkers include, but are not limited to polyaldehydes or other multifunctional reagents, e.g., divinyl sulfone.

According to one preferred embodiment, the crosslinker is a polyaldehyde.

As used herein, the terms “aldehydes”, used interchangeably with the terms “polyaldehyde”, formyl, or methanoyl group, means a molecule containing two or more aldehyde groups or their hydrates, or their acetals or hemiacetals, wherein the molecule is capable of performing as described herein and is capable of reacting with the albumin during the crosslinking as part of the invention. The term “polyaldehyde” is not used herein to mean a polymeric substance made by self-polymerizing an aldehyde monomer.

Aldehydes are amine reactive and can be used to cross-link proteins and/or cells containing an amine, for example proteins and/or cells present in whole blood. Examples of polyaldehydes include glyoxal, glutaraldehyde, adipaldehyde, succinaldehyde, and suberaldehyde.

Glutaraldehyde is a dialdehyde that can be used as an amine-reactive homobifunctional protein cross-linker. Monomeric glutaraldehyde can polymerize by an aldol condensation reaction yielding alpha,beta-unsaturated poly-glutaraldehyde.

Thus, according to one preferred embodiment, the polyaldehyde is glutaraldehyde.

As noted above, the present inventors have successfully prepared nanoparticles containing an external non-dyed albumin coating, to prevent coagulation of the nanoparticles.

In this case, the process described herein further includes adding a non-dyed albumin coating on the albumin core nanoparticles or on the NIR dyed core shell nanoparticles, by precipitating albumin thereon.

As noted above, the present inventors have successfully prepared nanoparticles containing a variety of bioactive particles, so as to enhance the detection capabilities of the particles, or to add therapeutic properties to the particles.

For example, the bioactive agent can be attached to the outer layer of the nanoparticles. In this case, the process described herein further includes binding at least one bioactive agent to an outer albumin layer of the albumin nanoparticles, by activation of at least one functional group on the albumin, this functional group being selected from carboxylates, amines, thiols and hydroxyls, thereby obtaining activated nanoparticles, followed by interaction of the activated nanoparticles with at least one bioactive agent, selected from a protein, a peptide, an antibody, an oligonucleotide or a drug.

According to one preferred embodiment of the invention, the bioactive agent is attached to the albumin via a spacer, and therefore according to this embodiment, there is an additional step of introducing a spacer molecule to adhere to the albumin, followed by linking of the bioactive agent.

In another example, the bioactive agent can be incorporated within the core of the nanoparticles, thereby forming a slow-released therapeutic agent. In this case, the process comprises adding at least one bioactive agent to the aqueous phase, thereby forming a physical complex of the albumin and the dye, containing at least one bioactive agent trapped within, wherein the bioactive agent is selected from a protein, a peptide, an antibody, an oligonucleotide or a drug.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXPERIMENTAL

### Materials and Methods

### Materials

The following analytical-grade chemicals were purchased from commercial sources and were used without further purification: 2-[2-[2-Chloro-3-[2-[1,3-dihydro-3,3-dimethyl-1-(4-sulfobutyl)-2H-indol-2-ylidene]ethylidene]-1 cyclohexen-1-yl]ethenyl]-3,3-dimethyl-1-(4-sulfobutyl)-3H-indolium inner salt sodium salt (IR-783), Human serum albumin (HSA), 4-mercaptobenzoic acid, N,N-dimethylformamide (DMF), 1-ethyl-3-(3-dimethylaminopropyl)carbodiimide (EDC), sepharose 4B, peanut agglutinin (PNA) and fluorescein isothiocyanate-conjugated peanut agglutinin (FITC-PNA), hematoxylin and eosin from Sigma (Rehovot, Israel); N-hydroxysulfosuccinimide (Sulfo-NHS) from Thermo Scientific, U.S.A; 2-morpholino ethanesulfonic acid (MES, pH 6) from Fisher Scientific, U.S.A; Tissue-Tek® O.C.T. from Sakura, Japan. Phosphate Buffered Saline (PBS), Minimum Essential Medium (MEM) eagle, McCoy's 5A medium and Dulbecco's modification of eagle's medium (DMEM), glutamine, penicillin and streptomycin from Bet Haemek, Israel; LS174t and HT29 tumor cell lines from American Type Culture Collection (ATCC); monoclonal anti-CEA (T86-66) and anti-TAG-72 (CC-49) antibodies were purified from the hybridoma supernatant at Alomone Labs, Israel; human bowel juice were obtained from Prof. A. Nissan, Surgery Department, Rabin Medical Center, Beilinson Hospital, Israel. Water was purified by passing deionized water through an Elgastat Spectrum reverse osmosis system (Elga Ltd., High Wycombe, UK).

### Synthesis of the NIR Fluorescent CANIR Dye

The cyanine dye chosen for use throughout this work is a previously synthesized carboxylic acid derivative obtained as a dark green powder by interacting the commercially available IR-783 with 4-mercaptobenzoic acid in DMF, as reported in the literature [Strekowski L. et al., J. Heterocycl. Chem. 2003, 40:913-916]. The CANIR dye is obtained in high yield (90%) and 100% purity as shown by elemental analysis, MS and H¹NMR. The carboxylic acid functionality was used for covalent attachment of the dye to the primary amino groups of gelatin via the carbodiimide activation method [Corem-Salkmon E. et al. International Journal of nanomedicine, 2011; 6:1595]. The CANIR-conjugated gelatin was then used for the preparation of the NIR fluorescent 10 nanoparticles.

### Synthesis of the NIR Fluorescent HSA Nanoparticles

The NIR fluorescent HSA nanoparticles were prepared by a desolvation technique via a precipitation process [Langer K. et al., Int. J. Pharm. 2003, 257:169-180; Weber C. et al. Int. J. Pharm., 2000, 194:91-102]. Briefly, ethanol (12.5 mL) was added to an aqueous solution (6.25 mL) of HSA (250 mg) and CANIR (1.25 mg). The obtained solution was then stirred at room temperature for 15 minutes at 800 rpm. Then, the temperature was raised to 70° C. and the mixture was stirred for additional 4 hours, followed by 78° C. for 12 hours. The obtained NIR fluorescent particles dispersed in water were then washed from impurities by sepharose 4B gel filtration.

Non-fluorescent HSA nanoparticles were prepared similarly to those of the NIR fluorescent HSA nanoparticles, in the absence of the CANIR dye.

### Determination of the Encapsulated CANIR Concentration in the NIR Fluorescent HSA Nanoparticles

The concentration of the encapsulated CANIR was determined for 0.5 mg/mL nanoparticles, via a calibration curve obtained by measuring the integral of absorbance peaks of standard solutions of free CANIR in PBS (at wavelengths 630-900 nm).

Optimization of the CANIR Concentration Encapsulated within the HSA Nanoparticles

Different concentrations of the CANIR dye were added to the 4% aqueous HSA solution (weight % ratio of the CANIR dye and the NIR fluorescent HSA nanoparticles were 0.1, 0.25, 0.5, 0.7 and 1), followed by the formation of the fluorescent nanoparticles. The NIR fluorescent HSA nanoparticles dispersed in PBS were diluted with PBS to 0.5 mg/mL and their fluorescence intensities at 823 nm were then measured.

Leakage Extent of the CANIR Encapsulated in the Fluorescent HSA Nanoparticles into PBS and Human Bowel Juice

4% HSA solution in PBS containing the NIR fluorescent HSA nanoparticle (1 mg/mL) was shaken at 37° C. for 4 hours and then filtered via a 300-kDa filtration tube (VS0241 VIVA SPIN) at 4000 rpm. The fluorescence intensity of the supernatant was then measured at 750 nm. A similar procedure was performed with human bowel juice as a continuous phase substituting the 4% HSA solution in PBS.

### Conjugation of the Tumor-Targeting Ligands (PNA, Anti-CEA and Anti-TAG-72 Antibodies) to the NIR Fluorescent HSA Nanoparticles

PNA was covalently conjugated to the NIR fluorescent HSA nanoparticles via the cabodiimide activation method [17]. Briefly, EDC (1 mg) and Sulfo-NHS (1 mg) were each dissolved in 0.1 M MES (pH 6.0, 1 mL) containing 0.5 M NaCl. The EDC solution (1 mg/mL, 10 mL) was added to an aqueous solution of PNA (0.25 mg, 62.5 mL), followed by the addition of the sulfo-NHS solution (1 mg/mL, 25 mL). The mixture was then shaken at room temperature for 15 minutes, followed by the addition of the NIR fluorescent HSA nanoparticles (2.5 mg, 1 mL PBS). The mixture was then shaken for an additional 90 minutes. The obtained PNA-conjugated fluorescent nanoparticles were then washed from excess reagents by sepharose 4B gel filtration. FITC-PNA, anti-CEA and anti-TAG-72 antibodies were conjugated to the NIR fluorescent HSA nanoparticles via a similar procedure substituting the PNA by the other bioactive agents. The concentration of bound PNA (2.4 μg/mg nanoparticles) was determined with FITC-PNA via a calibration curve of FITC. The concentration of bound anti-CEA and anti-TAG-72 antibodies (2.2±0.2 μg/mg nanoparticles) were determined by ELISA.

### Characterization of the NIR Fluorescent HSA Nanoparticles

Particle size and size distribution were determined by DLS with photon cross-correlation spectroscopy (Nanophox particle analyzer, Sympatec GmbH, Germany) and by Scanning Electron Microscopy (SEM) (JEOL, JSM-840 Model, Japan). For the SEM study, the diameter of more than 100 particles was measured with the image analysis software AnalySIS Auto (Soft Imaging SystemGmbH, Germany).

Fluorescence measurements of the FITC-PNA were performed using a multiplate reader TECAN SpectraFluor Plus (Neotec Scientific Instruments) with excitation wavelength of 485±10 nm and emission wavelength of 535±10 nm, integration time was 40 μs and gain was set to 70.

Fluorescence spectra of the non-conjugated and bioactive conjugated NIR fluorescent HSA nanoparticles were recorded using a spectrofluorometer (Cary eclipse, Agilent Technologies Inc.). Excitation and emission slits were fixed at 5 nm, and λex was set at 750 nm.

Photobleaching is the irreversible light-induced destruction of the fluorophore, affected by factors such as oxygen, oxidizing or reducing agents, temperature, exposure time and illumination levels. For photobleaching experiments, the samples were diluted to 0.05M and λex was set at 800 nm and λem at 830 nm. Each of the samples was illuminated continuously, and fluorescence intensity was measured over a period of 20 minutes. The intensity values were normalized.

### Chicken Chorioallantoic Membrane (CAM) Grafting Procedures

Tumor cells were grafted on CAM according to the literature. Briefly, fertile chicken eggs obtained from a commercial supplier were incubated at 37° C. at 60-70% humidity in a forced-draft incubator. At 3 days of incubation, an artificial air sac was established dropping the CAM. A window was opened in the shell and the CAM exposed on day 8 of incubation. Tumor cells were collected by trypsinization, washed with culture medium and pelleted by gentle centrifugation. After removing the medium, 5×10⁶ cells were resuspended in 30 μL ice-cold Matrigel and inoculated on the CAM at the site of the blood vessels. Eggs were then sealed and placed back into the incubator. On day 6 post-grafting (day 14 of incubation), the tumor size ranged from 3 to 5 mm in diameter with visible neoangiogenesis.

A chicken embryo CAM model has been used in this work for testing the specific tumor detection by both the non-conjugated and the bioactive (e.g., PNA and anti-CEA antibodies) conjugated NIR fluorescent HSA nanoparticles. All cancer cell lines evaluated in this study were able to form solid tumors, 3 to 5 mm in diameter depending on the cell line.

### Cell Lines

Human colorectal adenocarcinoma cell lines were used for each of the experiments. The LS174t cell line was maintained in Minimum Essential Medium (MEM) eagle supplemented with heat-inactivated FBS (10%), penicillin (100 IU/mL), streptomycin (100 μg/mL) and 1-glutamine (2 mM). The HT29 cell line was maintained in McCoy's 5A medium supplemented with FBS (10%), penicillin (100 IU/mL), streptomycin (100 μg/mL) and 1-glutamine (2 mM).

Detecting of Human Colon Tumor with the Non-Conjugated and Bioactive-Conjugated NIR Fluorescent HSA Nanoparticles in a Mouse In-Vivo Model

Experiments were performed according to protocols of the Israeli National Council for Animal Experiments by Harlan Biotech, Israel. Athymic nude mice (50 mice) with implanted 2-week old lumen-facing tumors (LS174t and HT29 cell lines) were anaesthetized and treated with non-conjugated and bioactive-conjugated NIR fluorescent HSA nanoparticles (0.1%, 200 μL), via the anus, using guidance of a mini-colonoscope. 20 minutes later each colon was washed with extensively with PBS (5×1 mL) and mice were allowed to recover for 4 hours. The mice were sacrificed and the colons were removed. Each colon was spread on a solid surface, and imaging was performed by using the Odyssey® Infrared Imaging System, (Li-Cor Biosciences, Lincoin, Nebr., USA) with excitation wavelength 780 nm and emission wavelength 800 nm, or using a Maestro II in-vivo fluorescence imaging system (Cambridge Research & Instrumentation, Inc., Woburn, Mass.). A NIR excitation/emission filter set was used for the experiments (λex: 700-770 nm, λem>790 nm). The Liquid Crystal Tunable Filter (LCTF) was programmed to acquire image cubes from A=790 nm to 860 nm with an increment of 10 nm per image.

### CEA Measurement:

CEA, a highly glycosylated glycoprotein, is highly expressed in most human carcinomas and therefore used as a marker in several modalities of human carcinoma management. According to the literature this antigen is upregulated on the mucosal side of various colorectal cancer cell lines such as LS174t colorectal cancer cell line, compared to the HT29 cell line in which this antigen expressed to a much lower extent [see: Kantor J. et al. Cancer research. 1989; 49:2651-5].

### Histological Analysis

Following the in vivo mice experiments the tissues were frozen in liquid nitrogen in Tissue-Tek® O.C.T. compound and cryosectioned at 5 microns in the cryostat. The sections were picked up on a glass slide and stained with hematoxylin and eosin (H&E) for histopathological examination. The sections were also spread on a stab, stained with H&E and visualized by SEM.

### Experiments and Results

### Synthesis of Dyed NIR HSA or NIR HSA-Coated Nanoparticles

### Example 1

### Synthesis of Albumin Nanoparticles Containing CANIR Dye

An aqueous solution (6.25 mL) of HSA (250 mg) and CANIR (1.25 mg) was prepared. Ethanol (12.5 mL) was added, and the mixture was stirred for 15 minutes at 800 rpm. The mixture was heated to 70° C. and stirred for a further 4 hours. The temperature was then increased to 78° C., for ethanol evaporation. Excess ethanol was removed by dialysis against water or PBS and the particles were further separated from impurities by crosslinked dextran gel filtration. The particles obtained were 100±15 nm in dry diameter, as shown in a SEM image. Concentrations and volumes of water, temperature, ethanol or an alternative organic solvent, HSA and CANIR are variable, for variation of particle size and fluorescence intensity. Besides this desolvation method, various other methods in addition of the NIR dye may also be used for synthesis of albumin particles, e.g., a pH-coacervation method, crosslinking with glutaraldehyde method or emulsification method [Langer, Balthasar et al., Int. J. Pharm., 2003, 257(1-2) 169-180].

A similar process was also accomplished for cancer drugs encapsulation, e.g., adriamycine, etc., or other bioactive agent/s, e.g., gen/s, antibiotics, contrast agents for CT and/or MRI, etc., within the albumin particles substituting the albumin solution containing the NIR dye for albumin solution containing the NIR dye and the drug/s and/or other bioactive agent/s.

To ensure that no CANIR remains on the surface of the particles, an additional coating of HSA may be applied. HSA (18 mg) was added to dyed albumin particles (90 mg). While shaking, the mixture was gradually brought to 70° C., and shaken for a further 16 hours before cooling. Any remaining HSA in solution was removed by dialysis against PBS or water. Concentrations, temperature, coating time and ratios of particles to HSA may be varied to give different coating thicknesses.

CANIR may be substituted by any of the dyes mentioned in sections 1.2 and 1.5. Similar results are obtained.

### Example 2

### Synthesis of Magnetic NIR Dyed Albumin Nanoparticles

An aqueous solution (5 mL) containing HSA (200 mg) and CANIR (1 mg) was prepared. Ferro-fluid (100 μL containing 5 mg iron oxide) was added, and the mixture was stirred for 15 minutes at 800 rpm. The mixture was heated to 70° C. and stirred for a further 4 hours. Excess reagents were removed by dialysis against water or PBS and the particles were further separated from impurities by a magnetic gradient column. Concentrations and volumes of water, ferro-fluid, HSA and CANIR are variable, for variation of particle size, magnetic properties and fluorescence intensity.

Besides this desolvation method, various other methods in addition of the NIR dye may also be used for synthesis of albumin particles, e.g., a pH-coacervation method, crosslinking, for example by the glutaraldehyde method or emulsification method [Langer, Balthasar et al., Int. J. Pharm., 2003, 257(1-2) 169-180]. Crosslinking is also possible using other polyaldehyde or other multifunctional reagents, e.g., divinyl sulfone.

A similar process was also accomplished for cancer drugs encapsulation, e.g., adriamycine, etc., or other bioactive agent/s, e.g., gen/s, antibiotics, contrast agents for CT and/or MRI, etc., within the albumin particles substituting the albumin solution containing the NIR dye and the magnetic ferro-fluid for albumin solution containing the NIR dye, the magnetic ferro-fluid and the drug/s and/or other bioactive agent/s.

An additional coating of HSA may be added, as in example 1. CANIR may be substituted by any of the dyes mentioned in sections 1.2 and 1.5. Similar results are obtained.

### Example 3

### Synthesis of Core-Shell NIR-HSA Coated Magnetic Iron Oxide Nanoparticles

Core Particles:

Iron oxide (10) core nanoparticles were prepared in 2 ways:

First way: Nanoparticles with narrow size distribution were prepared by nucleation followed by controlled growth of magnetic iron oxide thin films onto gelatin/iron oxide nuclei, as described in detail in EC 10880315 (2003); IL 139638 (2006). Briefly, an FeCl₂ solution (10 mmol/5 ml H₂O) was added to an aqueous solution (80 mL) of gelatin (200 mg) at 60° C., followed by a NaNO₂ solution (7 mmol/5 ml H₂O). The mixture was shaken at 60° C. for 10 minutes, and NaOH aqueous solution (1 N) was added until a pH of 9.5 was achieved. This procedure for growth of magnetic films onto gelatin nuclei was repeated four times. The formed magnetic nanoparticles were then washed from excess reagents using magnetic gradient columns. The particles formed were 15±1.2 nm in dry diameter, as shown in a TEM image. The number of cycles of growth of magnetic films onto the iron oxide nuclei may be varied to give different particle sizes and varying magnetic properties. NaOH may be replaced by an aqueous ammonia solution.

Second way: iron oxide magnetic nanoparticles of 17±2.5 nm dry diameter were prepared similarly to as described previously by Molday et al. [Molday and Mackenzie, J. Immunol. Methods, 1982, 52(3) 353-367]. Briefly, these nanoparticles were prepared by mixing 50% (w/w) dextran T-40 (10 mL) with an equal volume of an aqueous solution containing FeCl₃.6H₂O (1.51 grams) and FeCl₂.4H₂O (0.64 grams). While stirring, the mixture was titrated to pH 10-11 by the dropwise addition of 7.5% (v/v) NH₄OH and heated to 60° C. in a water bath for 15 minutes. Aggregates were then removed by centrifugation. The obtained iron oxide nanoparticles were then washed from unbound dextran using magnetic columns.

Albumin-Based Shell:

Magnetic iron oxide core particles (40 mg) were added to albumin (20 mg) and CANIR (2 mg) diluted to give a total concentration of 4 mg particles/mL water. While shaking, the mixture was gradually brought to 65° C., and shaken for a further 16 hours before cooling. Excess dye and any dissolved albumin remaining was removed using a magnetic gradient column. An additional coating of albumin was obtained by adding the NIR-magnetic nanoparticles particles (20 mg) to albumin (5 mg) and dilution to give a total concentration of 4 mg particles/mL. While shaking, the mixture was gradually brought to 65° C., and shaken for a further 16 hours before cooling. Any dissolved albumin remaining in solution was removed by a magnetic gradient column. Concentrations of dye and albumin and temperature and time were varied to give different coating thicknesses and fluorescence intensities. Similar coatings were obtained for both types of the magnetic iron oxide nanoparticles.

CANIR may be substituted by any of the dyes mentioned in sections 1.2 and 1.5. Similar results are obtained.

Albumin-based shell containing drug/s or other bioactive agents are prepared similarly substituting the CANIR for albumin solution containing CANIR and an appropriate drug, e.g., adriamycine or other bioactive agent/s as described in example 1.

### Example 4

### Synthesis of Core-Shell NIR-HSA Coated Dyed Iron Oxide Nanoparticles

Core Dyed Iron Oxide Nanoparticles:

Synthesis of dyed core particles was achieved similarly to that described for the non-dyed iron oxide nanoparticles (see example 3) substituting the gelatin or dextran for covalently dyed-conjugated gelatin or dextran. For example, covalent attachment of CANIR (or an alternative dye with a carboxylic acid group) to gelatin was accomplished by addition EDC (1 mg) and Sulfo-NHS (2.2 mg) to CANIR (or an alternative dye with a carboxylic acid group) (2 mg) in water (2 mL). The mixture was shaken for 15 minutes and added to an aqueous solution (80 mL) of gelatin (200 mg) at 60° C. NaOH (1N) was added until a pH of 8.5 was achieved. The mixture was shaken at 60° C. for 1 hour, and the pH was adjusted to 5 by addition of HCl (1N). This solution was used instead of the gelatin solution for synthesis of core magnetic particles, as in example 3.

A similar process was also accomplished for the preparation of nanoparticles containing a drug and iron oxide, substituting the gelatin or dextran for covalently drug conjugated gelatin or dextran.

Albumin-Based Shell:

The albumin-based shell was formed as in example 3. In this way both the core and the shell of the magnetic nanoparticles were NIR fluorescent dyed, so that the fluorescence intensity was significantly higher than that of the magnetic nanoparticles composed of NIR fluorescent core or shell only.

### Example 5

### Synthesis of Core-Shell NIR-HSA Coated Polystyrene Nanoparticles

### Polystyrene Core Particles:

Polystyrene core particles were synthesized by a single-step emulsion polymerization process. An aqueous solution (15 mL) containing K₂SO₃ (10 mg) and tween 20 (150 mg) was added to styrene (1.1 mL). The mixture was deaerated with nitrogen gas, and polymerized at 74° C. for 16 hours. The particles were dialysed extensively against water. Average particle size was measured by light scattering and found to be 105±15 nm in hydrodynamic diameter. Polystyrene particles of sizes ranging between 10-500 nm were prepared by changing various parameters in the emulsion polymerization process, e.g., polymerization temperature, monomer concentration, stabilizer type and concentration and crosslinker monomer type and concentration.

Albumin-Based Shell:

Polystyrene particles (20 mg) were added to albumin (10 mg) and CANIR (20 mg) diluted to give a total concentration of 1.5 mg particles/mL. While shaking, the mixture was gradually brought to 65° C., and shaken for a further 16 hours before cooling. Excess dye and any dissolved albumin remaining was removed using repeated centrifugation cycles or ultrafiltration. An additional coating of albumin was obtained by adding NIR-HSA dyed polystyrene particles (20 mg) to albumin (5 mg) and dilution to give a total concentration of 1.5 mg particles/mL. While shaking, the mixture was gradually brought to 65° C., and shaken for a further 16 hours before cooling. Any dissolved albumin remaining in solution was removed by ultrafiltration. HSA coating temperature, concentrations of dye and albumin were varied to give different coating thicknesses and fluorescence intensities.

CANIR may be substituted by any of the dyes mentioned in sections 1.2 and 1.5. Similar results are obtained.

### Example 6

### Synthesis of Core-Shell NIR-HSA Coated Polystyrene Nanoparticles of 200-3000 nm in Diameter

Core Particles:

Polystyrene (PS) nanoparticles of average diameters of 500 nm±10%) were synthesized by a single-step dispersion polymerization process. Benzoyl peroxide (5 mg) was dissolved in styrene (0.55 mL). A solution of PVP (MW 1000000, grams) in ethanol (15 mL) was added. The mixture was deaerated with nitrogen gas, and the mixture was polymerized at 74° C. for 16 hours while shaking. The particles obtained were washed with repeated centrifugation cycles with ethanol followed by water. Particle diameter was varied by varying monomer, initiator and stabilizer concentrations, as well as stabilizer molecular weight. The resulting particles were of average diameter 500±90 nm. Polystyrene particles of sizes ranging between 200-3000 nm were prepared by changing various parameters in the dispersion polymerization process, e.g., monomer concentration, stabilizer type and concentration and crosslinker monomer type and concentration.

Albumin-Based Shell:

The albumin-based shell was formed as in example 5 and excess reagents were washed using repeated centrifugation cycles.

### Example 7

### Synthesis of Core-Shell NIR-HSA Coated Dyed Polystyrene Nanoparticles, Below 500 nm in Diameter

Core Dyed Polystyrene Particles:

The dyed polystyrene core particles were synthesized by a single-step emulsion polymerization process. The NIR dye octabutoxy phthallocyanine (4 mg) was dissolved in styrene (1.1 mL). An aqueous solution (15 mL) containing K₂SO₃ (10 mg) and tween 20 (150 mg) was added. The mixture was deaerated with nitrogen gas, and polymerized at 74° C. for 16 hours. The particles were filtered through a 0.45 μm filter, and dialyzed extensively against water. The nanoparticles obtained were of 60 nm average diameter, as shown in a TEM image.

Core dyed polystyrene nanoparticles containing a bioactive agent, e.g., a cancer drug, were similarly prepared by substituting the polystyrene containing the dye with a combination of polystyrene containing the dye and an appropriate cancer drug.

### Albumin-Based Shell:

The albumin-based shell was formed and excess reagents were washed as in example 5.

### Example 8

### Synthesis of Core-Shell NIR-HSA Coated Dyed Polystyrene Nanoparticles (200-3000 nm in Diameter)

Core Dyed Particles:

Core polystyrene particles were synthesized as in example 6. The particle concentration was adjusted to give a 7% suspension. A solution of Octabutoxy phthalocyanine (4 mg) in DCM (1.75 mL) was prepared, a 1% (w/w) solution of SDS (10 mL) was added, and the mixture was sonicated for 4 mins creating an emulsion. The 7% particle suspension (3.5 mL) was added to the dye emulsion, and the mixture was shaken for 3 hours. The DCM was then allowed to evaporate, and the mixture was filtered through a 5 μm filter, removing insoluble, unencapsulated dye. Dye concentrations and SDS concentrations may be varied. Octabutoxy phthalocyanine may be replaced by other hydrophobic NIR-emitting dyes such as IR775, among others.

Albumin-Based Shell:

The albumin-based shell was formed as in example 5 and excess reagents were washed using repeated centrifugation cycles.

### Fluorescence Spectra of Particles:

The absorbance and emission of free dye in solution as well as encapsulated into nanoparticles were recorded. For each dye used, and for each type of particle, chromic shifts were observed in each of the spectra.

### Example 9

### Absorbance and Fluorescence Emission Spectra of Aqueous CANIR and CANIR-HSA Nanoparticles

In order to optimize the fluorescence intensity of the NIR fluorescent nanoparticles, different concentrations of the CANIR dye were added to the aqueous HSA solution, followed by the formation of the fluorescent nanoparticles via the precipitation process described above. The concentration of the entrapped CANIR dye that provided the maximum fluorescence intensity of the NIR fluorescent HSA nanoparticles was approximately 3.0 μg per 0.5 mg particles in 1 mL PBS. At higher dye concentrations, quenching of the fluorescence was observed, probably due to the closeness of the dye molecules entrapped within the nanoparticles, and thus non-emissive energy transfers between dye molecules.

Similar optimization of the fluorescence intensities was accomplished with the other dyes and the other types of fluorescent nanoparticles.

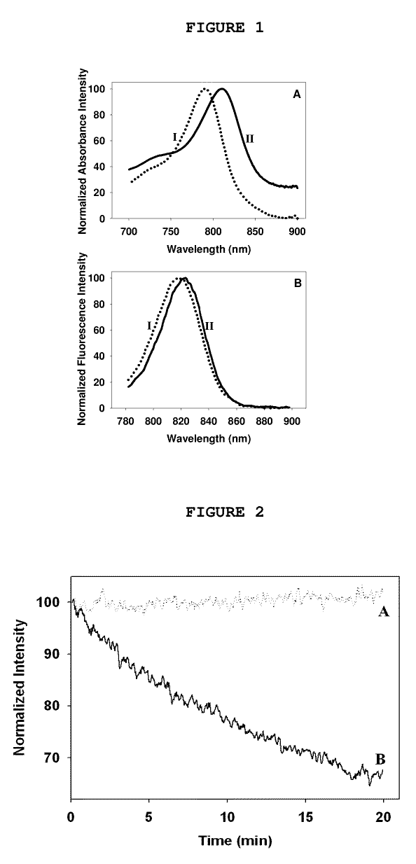

FIG. 1 exhibits the absorption (A) and fluorescence emission (B) spectra of free CANIR dye and the CANIR-HSA nanoparticles. The maximum absorption of free CANIR and NIR fluorescent HSA nanoparticles occurs at approximately 790 and 810 nm, respectively. The maximum fluorescence emission intensity occurs at approximately 818 and 823 nm, respectively. The red-shift of the NIR fluorescent HSA nanoparticles compared to free CANIR dye is probably due to physical binding to the HSA that affects the dipole moment of the dye.

### Example 10

### Stability Against Photobleaching Measurements of the NIR Fluorescent Nanoparticles

Photobleaching experiments were performed for the free CANIR dye and the CANIR-HSA nanoparticles, in order to examine their photostabillity. For the photobleaching experiments, the samples were diluted to 0.05M and λₑₓ was set at 800 nm and λₑₘ at 830 nm. Each of the samples was illuminated continuously, and fluorescence intensity was measured over a period of 20 minutes. Intensity values were normalized. FIG. 1 demonstrates that the encapsulation of the CANIR within the HSA nanoparticles reduces photobleaching. The encapsulation of the dye probably protects the dye against reactive oxygen species thereby reducing the photobleaching.

FIG. 2 depicts the photostability of the CANIR-HSA nanoparticles (A) and free CANIR (B) as a function of time. Samples of CANIR containing HSA nanoparticles and free CANIR were illuminated with a Xenon flash lamp for 20 minutes as described above. FIG. 2 demonstrates that, during illumination, the fluorescence intensity of the CANIR containing HSA nanoparticles remained almost unaltered while that of the free CANIR decreases significantly.

Similar stabilization behavior against photobleaching was also observed for the 10 core-shell NIR fluorescent nanoparticles and the polystyrene core-shell NIR fluorescent nanoparticles.

### Example 11

### Leakage Extent of the CANIR Encapsulated in the NIR Fluorescent Nanoparticles into PBS or Human Bowel Juice

4% HSA solution in PBS containing the NIR fluorescent HSA nanoparticles (1 mg/mL) was shaken at 37° C. for 4 hours and then filtered via a 300-kDa filtration tube (VS0241 VIVA SPIN) at 4000 rpm, or via magnetic columns wherein the nanoparticles possess magnetic properties. The fluorescence intensity of the supernatant was then measured at 750 nm. A similar trial was accomplished with human bowel juice as a continuous phase substituting the 4% HSA solution in PBS.

Leakage of the encapsulated CANIR dye into PBS dispersion in absence or presence of 4% soluble HSA was not detected following ultrafiltration as described above. Absence of leakage was also obtained wherein these fluorescent nanoparticles were dispersed in human bowel juice instead of PBS. These results indicate that the CANIR is strongly associated with the albumin nanoparticles as already known from the literature, so that free dye is not leaching into the continuous phase (PBS or bowl juice).

### Conjugation of Targeting Agents to the NIR Fluorescent Nanoparticles

Many possibilities to conjugated bioactive agents, e.g., via Shieff base formation, carbodiimide activation, Michael addition activation, etc., hereby just for demonstration a few conjugation methods are included, as follows:

### Example 12

### Conjugation of Targeting Agent/s to NIR-HSA Nanoparticles

Peanut agglutinin (PNA) was covalently conjugated to the surface of the dyed HSA nanoparticles by using the carbodiimide activation method. Briefly, 1-ethyl-3-(3-dimethylaminopropyl) carbodiimide (EDC) (1 mg) and N-hydroxysulfo-succinimide (Sulfo-NHS) (1 mg) were each dissolved in 0.1 M 2-[morpholino]ethanesulfonic acid (activation buffer at pH 6.0, MES, 1 mL) containing 0.5 M NaCl. EDC solution (10 μL) was added to an aqueous solution of PNA (0.25 mg, 62.5 μL) followed by addition of sulfo-NHS solution (25 μL). The mixture was shaken for 15 minutes and NIR albumin nanoparticles (2.5 mg, 1 mL PBS) were added. The mixture was shaken for further 90 minutes, and excess reagents were removed by sepharose gel filtration, or by magnetic gradient columns if magnetic albumin particles are used. The PNA may be replaced by fluorescein isothiocyanate-conjugated peanut agglutinin (FITC-PNA), EGF, uMUC-1, antiCEA, V8, antiTAG-72, etc.

The extent of binding of PNA-FITC was determined using a calibration curve of varying concentrations of FITC. The extent of binding of antiTAG-72 and V8 were determined by ELISA.

### Example 13

### Conjugation of Targeting Agent/s Via a Spacer to Core-Shell NIR-HSA Coated Dyed Iron Oxide Nanoparticles

Bifunctional PEG bisuccinimidyl ester (NHS-PEG-NHS, 5 mg) was added to NIR-PS-HSA nanoparticles (5 mg) in PBS (5 mL). The mixture was stirred for 20 minutes and excess NHS-PEG-NHS was removed by a magnetic gradient column. PNA (0.125 mg/0.125 mL PBS) was added and the mixture was further stirred for 1 hour, followed by addition of glycine (50 mg). Excess reagents were removed by a magnetic gradient column.

The PNA may be replaced by FITC-PNA, EGF, uMUC-1, antiCEA, V8, antiTAG-72, etc.

### Example 14

### Conjugation of Targeting Agent/s to NIR-PS-HSA Core-Shell Nanoparticles

Bifunctional PEG succinimidyl ester (5 mg) was added to NIR-PS-HSA nanoparticles (5 mg) in PBS (9.5 mL). The mixture was stirred for 50 minutes and PNA (0.5 mg/0.5 mL PBS) was added. The mixture was further stirred for 1 hour, glycine (50 mg) was added and stirred for an additional 20 minutes. The mixture was concentrated to a volume of 2 mL, and excess reagents were removed by sepharose gel filtration.

The PNA may be replaced by FITC-PNA, EGF, uMUC-1, antiCEA, V8, antiTAG-72, etc.

### Chicken Embryo “In Vivo” Model

### Example 15

### Optical Detection of Human Colon Tumors with NIR-HSA Nanoparticles on CAM Model

### Optical Detection of LS174t, HT29 and SW480 Colorectal Tumor Cell Lines by Non-Conjugated and PNA-Conjugated NIR Fluorescent HSA Nanoparticles

To demonstrate the feasibility of using the NIR fluorescent HSA nanoparticles for colon tumor detection, PNA was covalently conjugated to the nanoparticles. PNA binds to the terminal sugar β-D-galactosyl-(1-3)-N-acetyl-D-galactosamine of the Thomsen-Friedenreich antigen [30]. According to the literature, this antigen is upregulated on the mucosal side of various colorectal cancer cell lines such as LS174t and HT29, compared to the SW480 cell line in which this antigen expressed to a much lower extent [see: Sakuma S, Yano T, Masaoka Y, Kataoka M, Hiwatari K, Tachikawa H, et al. In vitro/in vivo biorecognition of lectin-immobilized fluorescent nanospheres for human colorectal cancer cells. Journal of Controlled Release. 2009; 134:2-10].

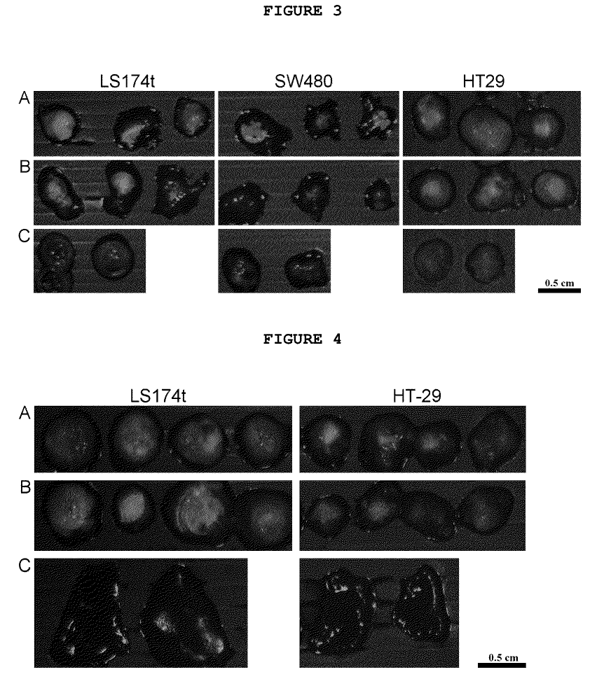

FIG. 3 exhibits the interaction between the non-conjugated (A) and the PNA-conjugated (B) fluorescent albumin nanoparticles and the human colonic cancerous cell lines: LS174t, HT29 and SW480 implanted on chicken CAM. FIG. 3 exhibits the specific detection of the colon tumor cell lines with the non-conjugated and the PNA-conjugated NIR fluorescent HSA nanoparticles. This figure illustrates that the fluorescence intensities of the LS174t and the HT29 tumor cell lines treated by the PNA-conjugated fluorescent HSA nanoparticles are 3-10 times higher than that of the SW480.

Similar results, but to a lesser extent, were observed for the non-conjugated NIR fluorescent HSA nanoparticles, e.g., the LS174t and HT29 cell lines were detected by these non-conjugated nanoparticles with fluorescence intensities 1-2 times higher than that of the SW480 cell line.

FIG. 3 shows that the LS174t and HT29 tumor cell lines were detected by the non-conjugated and the PNA-conjugated NIR fluorescent HSA nanoparticles with similar fluorescence intensities. On the other hand, this figure also shows that the fluorescence intensity of the SW480 tumor cell lines treated with the non-conjugate NIR fluorescent HSA nanoparticles was approximately 4 times higher than that treated by the PNA-conjugated nanoparticles. These results indicate that the SW480 tumor cell line responds to albumin significantly more than to PNA.

### Optical Detection of LS174t and HT29 Colorectal Tumor Cell Lines by Non-Conjugated and Anti-CEA Antibodies-Conjugated NIR Fluorescent HSA Nanoparticles

FIG. 4 exhibits the interaction between the non-conjugated (A) and anti-CEA antibodies-conjugated (B) fluorescent albumin nanoparticles and the human colonic cancerous cell lines: LS174t and HT29 implanted on chicken CAM. Non-pathological CAM treated with both the non-conjugated and the anti-CEA antibodies-conjugated NIR fluorescent HSA nanoparticles are shown in FIG. 4C.

FIG. 4 shows that the fluorescence intensity of the LS174t cell line treated with the anti-CEA antibodies-conjugated fluorescent albumin nanoparticles is 4-5 times higher than that of the HT29 cell line. Similar results, but to a lesser extent, are obtained for the non-conjugated albumin fluorescent nanoparticles, i.e., the fluorescence intensity of the LS174t interacted with the non-conjugated albumin nanoparticles is 1-2 times higher than that of the HT29 cell line. FIG. 4C shows that the fluorescence intensity of chicken CAM free of tumor implants treated with the non-conjugated or anti-CEA antibodies-conjugated fluorescent HSA nanoparticles is negligible, indicating the high specificity of these nanoparticles.

The particles used in this example may be replaced by any of the particles described in examples 1-8 with similar results.

### Example 16

### Optical Detection of Human Colon Tumors with Core-Shell IO NIR-HSA Nanoparticles on CAM Model

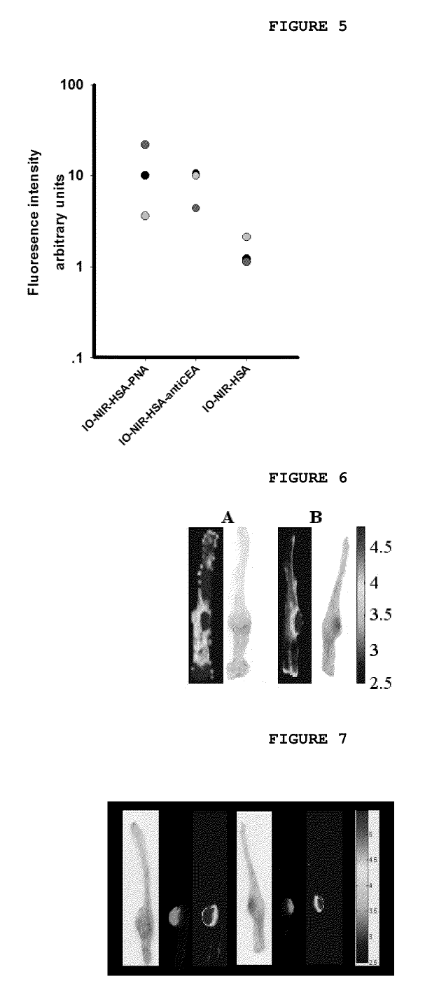

Chicken embryo CAM tumors were treated as in example 15, however the cell line used was LS-174 cancerous tumor cell line, and the particles used were core-shell unconjugated IO NIR-HSA nanoparticles (0.1%, 40 μL), and IO NIR-HSA nanoparticles conjugated to PNA or CEA (0.1%, 40 μL). ImageJ software was used for comparison of fluorescence intensity (FIG. 5). The particles used may be replaced by any of the particles described in examples 1-8. FIG. 5 shows the fluorescence intensities of specific labeling of tumor implants in chicken embryo with core-shell IO nanoparticles (IO-NIR-HSA). Since the scale is logarithmic, the differences in intensity between particles conjugated to a targeting agent and non-conjugated particles are significant.

### In Vivo Mouse Model

### Example 17

### Detecting of Human Colon Tumor with the Non-Conjugated and Bioactive-Conjugated NIR Fluorescent HSA Nanoparticles in a Mouse Model

For optimization of the tumor detection process, PNA was covalently conjugated to the nanoparticles as described above. PNA binds to the terminal sugar β-D-galactosyl-(1-3)-N-acetyl-D-galactosamine of the Thomsen-Friedenreich antigen (16). In vivo labeling of colonic neoplasms with PNA-conjugated nanoparticles was performed using a mouse model (6 mice) and LS174t tumor cell line. Mice were anesthetized and treated with a 0.1% PNA-conjugated nanoparticles dispersion in PBS, via the anus. 20 minutes later the colons were washed extensively with PBS and were allowed to recover for 1.5 hours (3 mice) and for 4 hours (3 mice). The mouse colons were removed and washed as described in the experimental part.

FIGS. 6 and 7 show color photographs and logarithmically scaled fluorescent images of LS174t tumor cell line from a typical mouse model treated with PNA-conjugated NIR fluorescent HSA nanoparticles, after a recovery of 1.5 h (FIG. 6A) and 4 h (FIG. 6B and FIG. 7). The colons were then removed and treated as described in the experimental part.

A typical fluorescence imaging picture shown in FIG. 6A exhibits that after 1.5 hours of recovery the tumor as well as other parts of the colon were labeled by the fluorescent PNA-conjugated nanoparticles. On the other hand, FIGS. 6B and 7 exhibits that after 4 hours of recovery the tumors were specifically labeled with a high signal to background ratio (SBR), the “background” being the surrounding non-pathological tissue. These results indicate that 4 hours of recovery is essential in order to self-wash well the non-specifically adsorbed fluorescent nanoparticles, thereby to increase the SBR.

Similar mice trials with 4 hours recovery time were accomplished with targeting agents other the PNA, e.g., monoclonal antibodies against CEA and against TAG-72. For this purpose these antibodies were conjugated to the NIR fluorescent albumin nanoparticles and tested their efficiency for tumor detection in LS174t and HT29 colorectal cancer cell lines. CEA, a highly glycosylated glycoprotein, and TAG-72, a human mucin (MUC1)-like glycoprotein complex, are both highly expressed in most human carcinomas, and therefore used as biomarkers in several modalities of human carcinoma. These two colon tumor cell lines are interested for the present study since the HT29 cell line expresses the CEA and TAG-72 antigens to a much lower extent, at least 10³ times, than the LS174t.

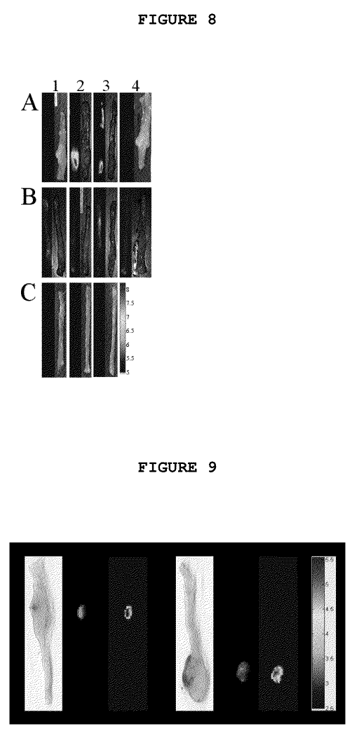

FIG. 8 shows fluorescent and grayscale images of LS174t (A) and HT29 (B) colon tumor cell lines treated with non-conjugated (1) and anti-CEA (2) and anti-TAG-72 (3) antibodies-conjugated NIR fluorescent HSA nanoparticles. This figure clearly illustrates that the non-conjugated nanoparticles did not significantly detect the tumors of both LS174t and HT29 cell lines.

In addition, FIG. 8C exhibits that the anti-CEA and anti-TAG-72 antibodies-conjugated NIR fluorescent HSA nanoparticles did not label the colons of the healthy mice. Also, FIGS. 8A4 and 8B4 indicate that under the experimental conditions the auto-fluorescence signal of the non-treated tumor cell lines is negligible.

It should also be noted that the mouse colon treated with the anti-TAG-72 antibodies-conjugated fluorescent HSA nanoparticles shown in FIG. 8A3 has two clear fluorescent signals (both with a SBR of approximately 50). The tumor in the bottom of the colon is visible to the eye, whereas the second fluorescent signal was only revealed as pathological subsequent to histological analysis. This result may indicate on the significant advantage of NIR fluorescence imaging using NIR fluorescent nanoparticles, over regular colonoscopy.

It should be noted that usually in the present mouse model the generated tumors were lumen-facing. However, infrequent this was not the case and the tumors were not faced to the lumen. FIG. 9 exhibits for example the histological analysis of lumen-facing and non-lumen-facing tumors. The presently prepared bioactive-conjugated NIR fluorescent HSA nanoparticles detect specifically the tumors faced to the lumen and not the ones that were not lumen-faced. These results demonstrate the specificity of the bioactive nanoparticles.

The tumor can be replaced with any human cancerous cell line. The nanoparticles used may also be replaced by any of the particles described in examples 1-8 and similar particles.

### Example 18

### Detection of Mouse Colon Tumors with CEA Antibodies-Conjugated NIR Iron Oxide-HSA Nanoparticles

Labeling of mouse colon tumor was performed similarly to example 17, replacing the bioactive-conjugated NIR-HSA nanoparticles with CEA antibodies-conjugated NIR iron oxide-HSA nanoparticles (FIG. 9).

FIG. 9 shows mouse colon tumors labeled with CEA antibodies-conjugated NIR iron-oxide-HSA nanoparticles. Two colons are displayed and each colon is depicted in a brightfield image, fluorescent image and a logarithmically scaled image. The tumors were selectively and effectively labeled by the nanoparticles, leaving surrounding tissue relatively unlabeled, with a high signal to background ratio.

The nanoparticles used may be replaced by any of the particles described in examples 1-8.

### Example 19

### Detection of Mouse Colon Tumors with Dyed Core-Shell NIR Fluorescent PS-HSA Nanoparticles

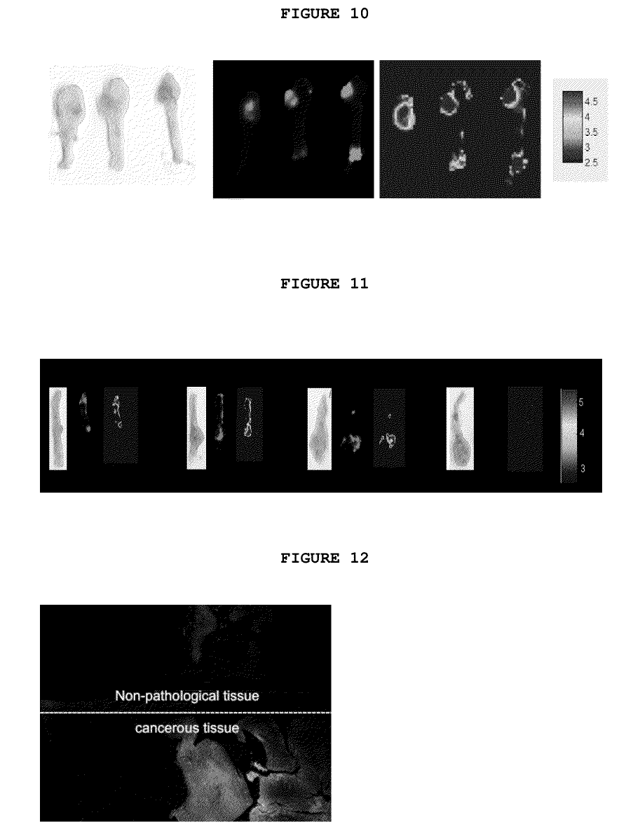

Labeling of mouse colon tumor was performed similarly to as in example 18, replacing CEA antibodies-conjugated NIR iron oxide-HSA nanoparticles with dyed core-shell non-conjugated NIR fluorescent PS-HSA nanoparticles (FIG. 10). FIG. 10 shows mouse colon tumors labeled with dyed core-shell NIR-HSA polystyrene nanoparticles. Three colons are displayed and depicted in a brightfield image, fluorescent image and a logarithmically scaled image. The NIR-HSA coated polystyrene particles selectively labeled the tumors, and left the surrounding non-pathological tissue relatively unlabeled, with a high signal to background ratio.

### Example 20

### Detection of Mouse Colon Tumors with Dyed Core-Shell PNA-Conjugated NIR Iron-Oxide-HSA Nanoparticles

Detection of mouse colon tumor was performed similarly to as in example 18, replacing CEA antibodies-conjugated NIR iron oxide-HSA nanoparticles with PNA-conjugated NIR iron-oxide-HSA nanoparticles (FIG. 11). FIG. 11 shows mouse colon tumors labeled with NIR iron-oxide nanoparticles conjugated to PNA. Four colons are displayed and each colon is depicted in a brightfield image, fluorescent image and a logarithmically scaled image indicating a high signal to background ratio. The particles selectively labeled the cancerous tissue, leaving surrounding non-pathological tissue relatively unlabeled. The fourth colon contained a tumor, but was not treated with nanoparticles, showing lack of autofluorescence of the tumor.

### Example 21

### Detection of Human Patholological Colon with V8-Conjugated NIR Iron-Oxide-HSA Nanoparticles

V8-conjugated NIR iron-oxide-HSA nanoparticles (0.05%, 0.5 mL) was dropped onto a fixed paraffin slide with cancerous and non-cancerous sample from a single human colon. 1 hour later the slide was washed with PBS, and then viewed using a Maestro™ in vivo imaging system (FIG. 12). FIG. 12 shows a fluorescent image of human pathological and non-pathological colon tissue on a microscope slide. The IO-NIR-HSA nanoparticles conjugated to V8 effectively and selectively labeled the human cancerous tissue.

1. Near Infrared (NIR) fluorescent albumin nanoparticles having a structure selected from:
a. A core structure, said core comprising at least one NIR dye encompassed within albumin nanoparticles and optionally comprising a dyed or non-dyed contrast agent;
b. A core-shell structure, said core comprising at least one material selected from a dyed or non-dyed metal or metal oxide, a dyed or non-dyed contrast agent, and a dyed or non-dyed organic compound having a hydrophilic surface, wherein said core is coated by a shell comprising one or more layers of albumin encompassing at least one NIR dye within it.

2. The NIR albumin nanoparticles of claim 1, wherein said NIR dye is a cyanine dye.

3. The NIR albumin nanoparticles of claim 2, wherein said cyanine dye is a dye absorbing in the range of 700-1000 nm.

4. The NIR albumin nanoparticles of claim 2, wherein said cyanine dye is selected from ICG, IR-820, IR-806, IR-783, IR-786, DTTCI, Cy7, cypate derivatives thereof and carboxylic acid derivatives thereof (CANIR).

5. The NIR albumin nanoparticles of claim 1, wherein said contrast agent is an X-ray contrast agent, or a CT contrast agent, selected from iron oxide, gold (Au), Barium compounds and Bismuth compounds.

6. The NIR albumin nanoparticles of claim 1, wherein said MRI-contrast agent is selected from iron oxide, Cobalt, Nickel and ferro-fluid.

7. The NIR albumin nanoparticles of claim 1, wherein said organic compound is an organic polymer.

8. The NIR albumin nanoparticles of claim 7, wherein said organic polymer is selected from polystyrene, poly(methyl methacrylate) (PMMA) and derivatives thereof.

9. The NIR albumin nanoparticles of claim 1, containing an additional non-dyed albumin external coating layer.

10. The NIR albumin nanoparticles of claim 1, having a diameter ranging from 1 nm to 1000 nm.

11. The NIR albumin nanoparticles of claim 1, further encompassing at least one bioactive agent, said bioactive agent being selected from a targeting agent, a drug and combinations thereof.

12. The NIR albumin nanoparticles of claim 11, wherein said targeting agent is selected from a protein, a peptide, an antibody, a small molecule, an oligonucleotide, a morpholino oligonucleotide, a peptide nucleic acid, or a drug.

13. The NIR albumin nanoparticles of claim 11, wherein said bioactive agent is selected from peanut agglutinin (PNA), EGF, uMUC-1, antiCEA, V8, antiTAG-72, TNF-related apoptosis-inducing ligand (TRAIL), folic acid, doxorubicin, methatroxate and taxol.

14. The NIR albumin nanoparticles of claim 11, wherein said bioactive agent is attached to said NIR albumin nanoparticles via a spacer molecule.

15. A process for the production of NIR albumin nanoparticles, said process comprising:
a. interacting at least one NIR dye with albumin, thereby forming a physical complex of said albumin and said dye, and
b. either precipitating said physical complex by the addition of a denaturating agent in an aqueous phase, or crosslinking said physical complex with a crosslinker.

16. The process of claim 15, wherein said precipitating is conducted at a temperature ranging from 30° C. to 100° C.

17. The process of claim 15, wherein said denaturating agent is an alcohol selected from ethanol, ethylene glycol, and mixtures thereof.

18. The process of claim 15, further containing in the aqueous continuous phase one or more contrast agents and/or drugs.

19. The process of claim 15, wherein said crosslinking is conducted at a temperature ranging from 4° C. to 100° C.

20. The process of claim 15, wherein said crosslinker is a polyaldehyde.

21. The process of claim 20, wherein said polyaldehyde is glutaraldehyde.

22. The process of claim 15, said process further including adding a non-dyed albumin coating on said albumin core nanoparticles or on said NIR dyed core shell nanoparticles, by precipitating albumin thereon.

23. The process of claim 15, said process further including binding at least one bioactive agent to an outer albumin layer of said albumin nanoparticles, by activation of at least one functional groups on said albumin, said functional group selected from carboxylates, amines, thiols and hydroxyls, thereby obtaining activated nanoparticles, followed by interaction of said activated nanoparticles with at least one bioactive agent, selected from a protein, a peptide, an antibody, an oligonucleotide or a drug.

24. The process of claim 15, said process comprising adding at least one bioactive agent to said aqueous phase, thereby forming a physical complex of said albumin and said dye, containing at least one bioactive agent trapped within, wherein said bioactive agent is selected from a protein, a peptide, an antibody, an oligonucleotide or a drug.

25. The process of claim 23, wherein said bioactive agent is selected from peanut agglutinin (PNA), EGF, uMUC-1, antiCEA, V8, antiTAG-72, TNF-related apoptosis-inducing ligand (TRAIL), folic acid, doxorubicin, methatroxate and taxol.

26. The process of claim 15, wherein said bioactive agent is attached to said albumin via a spacer.

27. A method of in-vivo detecting of pathology by collecting fluorescent light emitted from a tissue binded to the Near Infrared (NIR) fluorescent albumin nanoparticles of claim 1, said method comprising:
a) administering to a patient the Near Infrared (NIR) fluorescent albumin nanoparticles of claim 1,
b) administering to a patient an in-vivo sensing device, comprising at least one illumination source, an optical system and a light sensor;
c) illuminating in-vivo tissue external to the in-vivo sensing device; and
d) collecting fluorescent light reflected from the tissue onto said light sensor by using said optical system.

28. The method of claim 27, wherein said Near Infrared (NIR) fluorescent albumin nanoparticles contain at least one contrast agent, and said method further including using at least one more detection method, selected from magnetic resonance imaging (MRI), CT imaging, optical imaging, ultrasound imaging, paraCEST imaging or a combination thereof.

29. The method of claim 27, wherein said Near Infrared (NIR) fluorescent albumin nanoparticles contain at least one bioactive agent, said bioactive agent being a drug, and said method further comprising treating said pathology by releasing said drug of said Near Infrared (NIR) fluorescent albumin nanoparticles.

30. The method of claim 27, wherein said pathology is a pathology of cancer and cancer related diseases.

31. The method of claim 29, wherein said pathology is a pathology of cancer and cancer related diseases and said bioactive agent is a therapeutic agent used in the treatment or prevention of cancer, or in the alleviation of symptoms associated with cancer.

32. The method of claim 27, wherein administering said Near Infrared (NIR) fluorescent albumin nanoparticles is conducted orally.

33. The method of claim 27, wherein said Near Infrared (NIR) fluorescent albumin nanoparticles and/or said in-vivo sensing device are in a form of a capsule suitable for detecting pathology in the gastrointestinal (GI) tract during its passage through the GI tract.
